SEQUENCE LISTING (6 sequences)

SEQ ID NO: 1 - DNA, 1246 bp, synthetic construct
  source 1..1246: mol_type = other DNA
gaaccctttcaaacaaaacccacagcgatttctttgaaagaatgcctggactgtgctcagagctctcagattttctcaggaccaacgagccgccgcctcgcagataccactgtaaattacccagcgccttacgttcgtttctgattatttgcattgcagtgggtttgttaatgaaggacaagagtttagaaaaatcttttattttggaagttggacacgcaatcgaccccagcagtgttcctaaaaggagctatgccatttggatgggggaaaaatgagggggcggggaagtccatgttacatttaaaacaacaacagcaacaaattaaaagcaaaacaaaacaacaaaaaaaaggaagtggagcaggaggaggaggtggtggtggaggagagggaggaggggcagcagcagcggcagcagcagcaaggatggagtcaggacccgtcccagtcccgggtgagcgcttttggatctgggttctggctcaacccacgtaatgccccaatctaaagttttctacgtgtgtgtggataggaaagtctaaatagagtttatcagaacccaggtggtcattctctacactctctctagcttggacccgaagaacaaaggcacatgagaggagaaaggtgaccagctggacgatgacggtcacgtccaaacggatcccccttgtcctcggctctctcgtcctgagagtgggtttaatttttttttttttaatttatataagaagggagctcttaaaggagagccatctccaccctcaggtatccatcacccagctccagcgcgcctgggaaaaccgccccgaaagtccaagaggaagctcagagttgtaacggccgcggagccagctcggcggtgacgcaaggtccagtccagattgccaggcccggggcatgagagaggatccttgtaggtttcggaggtgggggggctgcactccattgttcactccgggccaatcagggttggcccacttcctcccagccaatctcccttcacccccagcctccaacccaacccaccccgcccatcagcccctggatccccatcacctcccccgcatccccggcagttctggggaagcttcgtgacgccacaggtcccgcccccagctccggcccggggctagtgcgtgttgacgtcatgctgcgtgcgggccggtgcggaatcgctccttcaactccgcggggcagtaggagttagttagcaaagagccgaggccgggcgcgcgaccct

SEQ ID NO: 2 - DNA, 1140 bp, synthetic construct
  source 1..1140: mol_type = other DNA
ggggcgggcgcgcgggccggggccgcgggcgtggggcgcttaccggcggcgtgggcaggcaggcccgccggatggcctcggagctggagtgcagcgacgggtacttgtgctcagggagggtgggatgcatggcaaagtgaggctgcttgctgttcatggacatcatcgtggcggcttggcatgtatatccacaaacactccgaaagtccgcgggaaagtgcgtacgccggctcacccggcctcccttcggaggctgcagccgcggcggtcgcggcggctggcggcggccccgccgcgggctgctgctgctgctcctgctgctgccaggcgctccctctgaccgcgcagagcgccgcgcgccggcctcgcggtcccgcttctccgacagctctagccccgcgcgccgacgggatgcactcctctaacacctgagccccacttctcgcggccgtcccggggagctctcgcgagagctcgcggccccaccgcgctgacaggcatcagctgtctccgtctgtctgacgcgcgctctccttctcggcggccccgtgcgtctgcgcgcgcgcgcgctcgtccggccgcgaccagcgcgtgggagccgctcttatagtgaccaccagcaaggacagcgcaggtgatgcacctgtagctacccgggcatgcgcactgcccgcctcacctttcccaccgtggtctggaaagatcagaaggtccagctattgtctaagggtgggcaccttttagaataaaaaagacgagatgcgcgcacgcccctctatgtgtgtgtgtgtgtgtgtgtgtgcgtgcgtgtgtgtgtatgtgtgtatgtttcttctccctgatagcaagccttgaatatatgagcattacttaggcattcctcctcttcgggatgcctaacaagctgttgtataatgtatacagctggacatttctacgttaaattatgcccgcgcattcctctgtatacagtataaataacaacacagaaatgcagaaggtgctgtcttttgctgcaagaacctgtttctttaaaggtttataaataggttcctggagaataatcgcggtgacagacatttgtttgaagcagtcagacgctattgatttccatataatttaatttcctccgcatacttt

SEQ ID NO: 3 - DNA, 1119 bp, synthetic construct
  source 1..1119: mol_type = other DNA
ttgcatgtcattccttcccccaagtggaactcacctcagtgacacaggacgttagtaggaataacggtgatgactaataagcatcagtctaggtgtggcagtcttcctacggggcttaataaagcagaggtaccatcccgcattttgaaaatgacatcctgaaagagaaaccaagactgtctaaccagcagcagctcacagtccagactgaaagggggcactgagactctccacacctgacactcgtcttcagttgataccacgggtttgactccgtcctgtctcgggcgacgcccgtaagacaggtcgacactaccccgacgaaacaaacaaactggtttctgagccccgtagaaacggcggggtgcagcagggggagtgcctccgttaccgcctctccaacttcacagaggtcgttgctagtggccacggcggggtggggacgggcggcgttcgggactgggaagctacttgactgccacgtgctcctgcctcgcaacagcggctctagattccgagttatggggacaactgctcgagaggccagggtagggtaaggactccgcaacctagggtcccaaaaccctggagcgaatgcggactaagggagagcggccggcgcgtcaccggcgctggcgtgtgacgtcagcgcgtggaaggcggcacgcagcggaggcggctgggaaggagcagtgaggctgggctagaaccgtagggtcgggcggggtgctgtgcgaaggggcgggcggggtgctgtgcgaagggacgggaggggcgggctctgcaggcgggaagacagaaaggacgagacgggagagatgaaaaggatggagagagtgtaaagctgttaagagtcatgcagagatgcgggagagaactcactagttgaaaccaatttggaatataaagcatataaaattttcccacagctactacaccgagaaagggacttttttattttacatgaaagcgcactacttagggatgccacattttaaagagcaattttgctttaaaaagcaagtcttttcctattatatcttggatggttgaaacagaagaatgtttaagtcttgagatatttttaaaaagctgtatggggttttctacacctttaca

SEQ ID NO: 4 - DNA, 530 bp, synthetic construct
  source 1..530: mol_type = other DNA
gtggctgaatgtgaaatagcaaaggtatctgacttataagttgctgagagaaatgattccatacctaaaccctgcccccaactctaaaaacttcctgcttgatgttcatcaataatgaatggcccatccctgcaatactcagagggagcttacattgcacttttgagcagccggttagtaatgtacagtggatagagcctttctttcaaatcccagggcagtgggtccttcaaggctagcattgcattttcaattagctgctgagtgaatgcttggcatgtgtattagtgaagaggcacacaattagcgtattgttcctttctgtattgtgctgagaggatccaagggattggtgggggaacaggcaagccaggcatcaccgtggatgttgaagaagggggttactcaaacctcggcatcttcacttgctcgatctggaattacagctatttattacgaagcactctgtgtggcttagtggagtgtgtctgaggaaacacatcccggacaccacttagggttagtctttc

SEQ ID NO: 5 - DNA, 1046 bp, synthetic construct
  source 1..1046: mol_type = other DNA
gtatacatgtgagggcttatgcagacacgactgtatacatgcatgtgtgaatgtgtacatatgcgcgtttgaatgtccggatcacatatgtgcccgtgtgcatgcctggagaggtgcatgtgtgggacagtcaggtcggcaggagtgcatgagaacggtgtgggcacacgtaagtgcacgatcacacatacaagtgagcttgagagtgtgtattcctgtgcactgtgtgcacacctgtgaccccttcaaacccctcatttgagaatcaaatgatgtcggaatggactccaccttgaatgtcgatggccgaggtaagggcgcctgcgcccctcccctccccctaggcctctgcccagggatctcctgcactgggaaggggaggggtggggatggggtggaggtggggtgatggactgagatgaagggcagggagggcaagcgggcattcttactgatagggggcagttagggagcaaggctccagggagaaggtgaagccagaggcggaggaagatgggtaagagagtgaaccctgtggcgggccgcagccggagaggaacaggaaccgcagtggggacgccctggtccccgggcccacagcatccgggacggacgcgcagtagcgcgggccgggaactgggtaccagggcgggatgggtgagaggctctaagggacaaggcagggagaagcgcagcggggtgcggggaaccgcacgccctccctttgcctctgcttcccaccccgaggcggcagggcgggcgggcgcggttccgggggtgggcgggctgggcggggcggaggcggggccgcagcactggcttcacccagcctctcccgcccgcagccagagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgagtgt

SEQ ID NO: 6 - DNA, 996 bp, synthetic construct
  source 1..996: mol_type = other DNA
accatctccgccaggcagcttggcaaacaggtggcagagttgcagggcagctgtgtaagccaacttcggcgcagcagtggagggtcctggcttggcgtgggggatgctggacccgcggtcgaggatttggggatataggggaagagggaggaggtggatgctgagccttgtatgcaggctatgtcagtttagccctctccccaacctctcttcggctcctgcccgtcccagaggagtgaggtggagaagggctggctgccagactggcaccaaaacagccttctttgggtgcccaggttgccagggctcgaggggtcggaggatatccagggaagcaccccaggtggtccaaaaagatcaaattttgaggacccctccctccccttttccctccccccccctccttccctgccgtgggctctttcagctgtggtccctttagaacccaggactactactgctcaacctcgctgggggttcgggtggctggattcgggtccctcactggcgtgacaggagggagtgcgaggcaggaatttaggagccaaggaggtgagagcagctctggcccctcactgtaggtgacgccaaactctcctcgacttgccccgactcttagttgaaaaatctctgtcctctcccaggctctccagcttcccaagcaatgacctcaatgaaaaaaatgacagcggggcggactgcccccgctccagagtaccagtgccggcagtgcgagctatgacgcaatcggagctcggtcggtcctttgattggctagtcctggccactttggattggccgcgcgggctggtggggaccccccccctccagctatctctgtaataagagcggggtctccgcggggaaggcgcccacagcaggtgtggtgttcatcccgggtcgaccggccgctcgcgctgccctgaaactctagtcgccaggtgaggaacttcccattccccattccgctcactcgaataacctgcttcttcgact

(19) United States
(12) Patent Application Publication
(10) Pub. No.: US 2026/0224750 A1
(43) Pub. Date: Aug. 6, 2026

(54) GENE THERAPY FOR TREATMENT OF PAIN

(71) Applicant: The General Hospital Corporation, Boston, MA (US)

(72) Inventors: Brian J. Wainger, Newton, MA (US); Aaron Held, Stoneham, MA (US); Daniel DuBreuil, Hudson, MA (US)

(21) Appl. No.: 19/159,178

(22) PCT Filed: Feb. 23, 2024
(86) PCT No.: PCT/US2024/017131

§ 371 Date: Aug. 22, 2025

Related U.S. Application Data
(60) Provisional application No. 63/486,864, filed on Feb. 24, 2023.

(51) Int. Cl. A61K 48/00; A61K 38/17; A61P 29/00; C07K 14/705; C12N 15/86

(52) U.S. Cl. CPC A61K 48/0058; A61K 38/177; A61P 29/00; C07K 14/705; C12N 15/86; C12N 2750/14143; C12N 2830/008

(57) ABSTRACT

Provided herein are methods and compositions for gene therapy for treatment of pain by increasing expression of potassium channels, preferably two-pore potassium channels, in nociceptors. Also provided are compositions and methods using short Promoters (SProms) that drive expression in sensory neurons.

### CLAIM OF PRIORITY

This application claims the benefit of U.S. Provisional Patent Application Ser. No. 63/486,864, filed on Feb. 24, 2023. The entire contents of the foregoing are hereby incorporated by reference.

### FEDERALLY SPONSORED RESEARCH OR DEVELOPMENT

This invention was made with Government support under Grant No. AG075419 awarded by the National Institutes of Health. The Government has certain rights in the invention.

### TECHNICAL FIELD

Provided herein are methods and compositions for gene therapy for treatment of pain by increasing expression of potassium channels, preferably two-pore potassium channels, in nociceptors. Also provided are compositions and methods using short Promoters (SProms) that drive expression in sensory neurons.

### BACKGROUND

Chronic pain plagues one quarter of adults in the US, inflicts a $500 billion annual economic burden, and fuels the opioid epidemic^{1,2}.

### SUMMARY

A method of treating pain in a subject, the method comprising administering to the subject a nucleic acid encoding a two-pore potassium channel, optionally wherein the two-pore potassium channel is KCNK2, KCNK4, or KCNK18, preferably KCNK4. In some embodiments, the two-pore potassium channel is not KCNK18.

In some embodiments, the nucleic acid encoding a two-pore potassium channel is administered by direct local injection into or near a site of pain, or into or near a nerve, plexus, dorsal root ganglia, or roots innervating the site of pain, in the subject. “Near” means within 6, 5, 4, 3, 2, or 1 inch of the site or the nerve.

In some embodiments, the nucleic acid encoding a two-pore potassium channel is administered is administered by epidural, intrathecal, intracisternal, intracerebroventricular, or systemic administration to the subject to the subject.

In some embodiments, the nucleic acid encoding a two-pore potassium channel is administered as DNA (e.g., a cDNA) or in a viral vector.

In some embodiments, the DNA or viral vector further comprises a promoter that drives expression of the two-pore potassium channel.

In some embodiments, the promoter is a ubiquitous promoter or a tissue specific promoter that preferentially drives expression in neurons, preferably in sensory neurons, preferably in nociceptors, preferably a synapsin promoter or a promoter listed in Table B or as shown in SEQ ID NOs: 1-6, or comprising a sequence at least 80% identical to a sequence provided herein, e.g., shown in Table B or as shown in SEQ ID NOs: 1-6. In some embodiments, the promoter is ISL2 SProm.

In some embodiments, the viral vector is an AAV, preferably AAV5, AAV6, or AAV9.

In some embodiments, the nucleic acid encoding a two-pore potassium channel is administered as mRNA, preferably in a composition comprising a deliver vehicle, optionally a lipid nanoparticle.

Also provided herein are nucleic acids comprising a sequence at least 80% identical to a sequence provided herein, e.g., shown in Table B or as shown in SEQ ID NOs: 1-6, optionally linked to a nucleic acid encoding a heterologous polypeptide for expression of the heterologous polypeptide.

Additionally, provided herein are expression vectors comprising a sequence at least 80% identical to a sequence provided herein, e.g., shown in Table B or as shown in SEQ ID NOs: 1-6, optionally linked to a nucleic acid encoding a heterologous polypeptide for expression of the heterologous polypeptide.

Further, provided herein are expression vectors comprising a promoter that preferentially drives expression in sensory neurons linked to a sequence encoding a two-pore potassium channel (i.e., that drives expression in sensory neurons, with substantially less or not detectable expression in other cell types).

In some embodiments, the viral vector is an AAV, preferably AAV5, AAV6, or AAV9.

In some embodiments, the heterologous polypeptide is a potassium channel, preferably a two-pore potassium channel. In some embodiments, the two-pore potassium channel is KCNK2, KCNK4, or KCNK18, preferably KCNK4 or KCNK18. In some embodiments, the two-pore potassium channel is not KCNK18.

Also provided are the nucleic acids or expression vectors described herein, comprising a sequence at least 80% identical to a sequence provided herein, e.g., shown in Table B or as shown in SEQ ID NOs: 1-6, optionally linked to a nucleic acid encoding a heterologous polypeptide for expression of the heterologous polypeptide, e.g., nucleic acids encoding a two-pore potassium channel, optionally wherein the two-pore potassium channel is KCNK2, KCNK4, or KCNK18, preferably KCNK4 or KCNK18, for use in a method of treating pain in a subject. In some embodiments, the two-pore potassium channel is not KCNK18.

Additionally provided herein are cells comprising the nucleic acids or expression vectors described herein, and optionally expressing the heterologous polypeptide.

Further, provided herein are AAV vectors comprising a nucleic acid encoding a KCNK4 potassium channel, linked to a promoter for expression of the KCNK4 potassium channel in sensory neurons. In some embodiments, the AAV vector is an AAV9 vector. In some embodiments, the AAV vector comprises a synapsin promoter or a promoter listed in Table B or as shown in SEQ ID NOs: 1-6, or comprising a sequence at least 80% identical to a sequence provided herein, e.g., shown in Table B or as shown in SEQ ID NOs: 1-6. In some embodiments, the promoter is ISL2 SProm, or comprises a sequence at least 80% identical to an ISL2 SProm sequence provided herein, e.g., shown in Table B or as shown in SEQ ID NOs: 1-6. Also provided herein are AAV vectors comprising a nucleic acid encoding a KCNK18 potassium channel, linked to a promoter for expression of the KCNK18 potassium channel in sensory neurons, wherein the promoter comprises a sequence at least 80% identical to a sequence provided herein, e.g., shown in Table B or as shown in SEQ ID NOs: 1-6. In some embodiments, the promoter is ISL2 SProm, or comprises a sequence at least 80% identical to an ISL2 SProm sequence provided herein, e.g., shown in Table B or as shown in SEQ ID NOs: 1-6. In some embodiments, the AAV vector is an AAV9 vector.

Also provided herein are methods to prevent or treat pain in a patient comprised of administering to said patient a therapeutically effective amount of any of the compositions described herein. In some embodiments, the composition is administered by, but no limited to, injection. In some embodiments, the injection is intramuscular or subcutaneous. In some embodiments, the injection is directed to a dermatome or other site-specific targeted region corresponding to a specific population of nociceptors. In some embodiments, the targeted site will include areas corresponding to, but not limited to, chronic post-surgical pain and neuroma, trigeminal neuralgia, post-herpetic neuralgia (shingles), focal cancer pain due to metastases, painful neuropathy (including chemotherapeutic neuropathy, diabetic neuropathy, and others), focal arthritis (joint injection), chronic back pain with sciatica or prophylactic pre-surgical treatment for high-risk procedures or high-risk patients.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Transduction of KCNK4 yielded lower nociceptor excitability compared to KCNA1 or KCNQ2. A, DRG were transduced in vitro with AAV9 viral particles containing either KCNA1 driven by a synapsin promoter (Syn-KCNA1) or KCNK4 driven by a synapsin promoter (Syn-KCNK4) and assessed via optical rheobase. Both viruses yield reduced excitability compared to controls in DuBreuil et al. Cell Rep Methods. 2021 May 24; 1(1): 100004. B, Comparison of candidate ion channels reveals substantial benefit for KCNK4. TrpV1::ChR2 mice underwent in vivo intrasciatic nerve AAV K channel injection. 4 weeks later, DRG were isolated and analyzed using the optical rheobase assay. Notably, KCNK4 yielded a marked reduction in nociceptor excitability compared to KCNQ2 and KCNA1. All graphs presented herein are mean±/−SEM.

FIG. 2. In vivo data in individual mice show AAV9 Syn-KCNK4 yielded a strong increase in mechanical withdrawal threshold. AAV9 viral particles containing Syn-potassium channels injected into the unilateral sciatic nerves yielded increased mechanical threshold in the ipsilateral versus contralateral side, compared to mice injected with Syn::GFP or untreated animals. The effect was larger for KCNK4 than KCNA1.

FIG. 3. Trigeminal Nociceptors. KCNK channels decreased optical threshold for nociceptor activation in the trigeminal ganglion. TrpV1::ChR2 trigeminal nociceptors ipsilateral to in vivo Syn-GFP-KCNK4, Syn-GFP-KCNK10, and Syn-GFP-KCNK18 trigeminal ganglion injection showed reduced excitability compared to trigeminal nociceptors ipsilateral to control AAV9 Syn-GFP injection. Trigeminal ganglia isolation and dissociation were performed four weeks following in vivo injection. Methods described in DuBreuil et al. Cell Rep Methods. 2021 May 24; 1(1): 100004.

FIG. 4. Dorsal Root Ganglion Nociceptors KCNK4 channel decreased optical threshold for nociceptor activation in the dorsal root ganglion (DRG). TrpV1::ChR2 nociceptors ipsilateral to in vivo AAV9 Syn-KCNK4 intrasciatic nerve injection show reduced excitability compared to contralateral control neurons (and separate Syn-GFP controls not shown). Methods described in DuBreuil et al. Cell Rep Methods. 2021 May 24; 1(1): 100004.

FIGS. 5A-B. Patch clamp of ex vivo nociceptors following in vivo intrasciatic nerve injection. Adult mice underwent intrasciatic nerve injection of AAV9 GFP control or AAV9 Syn-GFP-KCNK4 viral particles. 3-4 weeks afterwards, DRG were harvested and dissociated. Nociceptors were identified by size (capacitance <20 pF). A, panels show reduced firing (increased rheobase current) for GFP+ nociceptors from GFP-KCNK4-injected mice compared to GFP control. B, quantification shows increased rheobase, reduced input resistance, and hyperpolarized resting membrane potential in KCNK4 (grey, right hand bars) compared to GFP (open, left hand bars) nociceptors. These findings are all consistent with expected physiological function of KCNK4 channels and reduction of nociceptor excitability.

FIG. 6. Spared Nerve Injury Model. Intrasciatic nerve injection of AAV9 viral particles containing Syn-KCNK4 reduced pain in a rodent painful spared nerve injury model. Pain score of mechanical allodynia is decreased by AAV9-KCNK4 expression compared to AAV9-GFP control in a spared tibial nerve injury model. Spared tibial nerve model (Shields et al 2003) and proximal injection of viral particles were performed at day 0 and behavioral assessments initiated at week 3.

FIG. 7. Neuroma Model. Syn-KCNK4 gene therapy reduced pain in a rodent painful neuroma model. Animals underwent nerve transection and neuroma induction (as in Dorsi et al 2008) and concomitant proximal intrasciatic injection with AAV9 viral particles containing either Syn-KCNK4 or Syn-GFP. Pain score of mechanical allodynia (Dorsi et al. 2008) were decreased by Syn-KCNK4 expression compared to AAV9-GFP control. Compared to pre-surgical baseline pain composite scores, which are consistently near 0, both groups showed increased pain initially. However, animals treated with Syn-KCNK4 showed markedly decreased pain scores at 5 and 7 weeks.

FIG. 8. Inflammatory Pain Model. Syn-KCNK4 gene therapy reduced pain in a rodent inflammatory pain model. Pain score of thermal hyperalgesia is decreased by Syn-KCNK4 compared to Syn-GFP control. Four weeks after intrasciatic injection of AAV9 viral particles, CFA was injected in the ipsilateral paw. Whereas animals injected with Syn-GFP control showed marked thermal hyperalgesia on the ipsilateral compared to contralateral hindpaw, animals injected with Syn-KCNK4 viral particles showed minimal difference.

FIGS. 9A-B. POU4F1 and ISL2 promoters drive GFP vector expression in primary mouse sensory neurons in vitro. Expression of GFP in mouse primary DRG neurons 14 days after in vitro transduction with AAV9 vectors containing POU4F1 (a) or ISL2 (b) promoter short segments.

FIG. 10. Additional human SProms yielded DRG expression in nucleofected primary mouse DRG neurons. Short promoters segments from the human CALCB, NGFR, NEK1, and PP1R1C genes drive expression in nucleofected primary mouse DRG neurons.

FIGS. 11A-C. Intraplantar injection of AAV9 viral particles containing SProm-GFP transduced ipsilateral but not contralateral DRG. AAV9 viral particles containing SProm POU4F1-EGFP or SProm ISL2-EGFP were injected unilaterally into the plantar hindpaw. Several weeks later, ipsilateral and contralateral DRG were isolated, dissociated, and imaged for GFP expression. GFP is detected in the ipsilateral but not contralateral DRG neurons.

FIG. 12. Intrasciatic injection of ISL2 SPROM-GFP viral particles yielded ipsilateral DRG but not spinal cord expression. Whereas non-specific promoters have been shown to transduce both spinal motor neurons and DRG neurons after sciatic nerve injection, the ISL2 SProm promoter yielded robust DRG expression (left), but little to no expression in the ventral horn, including in spinal motor neurons (right), after intrasciatic nerve injection, thus demonstrating both the sensitivity and specificity of the SProm.

FIG. 13. ISL2 SPROM-GFP yielded GFP reporter expression in ipsilateral DRG neurons that included IB4-positive peptidergic nociceptors. After in vivo injection of AAV9 viral particles containing SProm ISL2-GFP, confocal microscopy of DRG sections shows GFP expression in cells that include IB4-positive non-peptidergic nociceptors.

FIG. 14. ISL2 SPROM-KCNK4 reduced nociceptor excitability. TrpV1::ChR2 mice underwent unilateral intrasciatic injection with AAV9 viral particles containing SProm ISL2-GFP-KCNK4. After sacrifice and DRG harvest 4 weeks later, ipsilateral TrpV1-positive nociceptors show reduced excitability compared to contralateral control TrpV1-positive nociceptors.

FIG. 15. Patch clamp of ex vivo trigeminal ganglion nociceptors following in vivo intraganglionic injection. After in vivo injection of AAV9 viral particles containing SProm ISL2-GFP-KCNK4, GFP in live imaging was too weak to identify transduced neurons. Blind patch clamp experiments showed many cells reflecting reduced excitability (increased rheobase and hyperpolarized resting membrane potential (RMP)) in ISL2-GFP-KCNK4 mice compared to control.

FIG. 16. Nerve Injury Model. Combination of ISL2 SPROM and KCNK4 within AAV9 viral particles yielded reduced pain in nerve injury model. AAV9 viral particles containing either SPROM ISL2-KCNK4 or SPROM ISL2-GFP control were injected into mouse unilateral sciatic nerves proximal to painful neuroma model induction. Mechanical hyperalgesia was reduced in animals injected with KCNK4 compared to GFP control viral particles.

FIG. 17. Intrasciatic injection of AAV Isl2-KCNK4 or Syn-KCNK4 each reduced thermal pain compared to control Isl-GFP or Syn-GFP following in the intraplantar CFA pain model. n=8 mice/group with equal number of adult male and female animals.

FIG. 18. Nerve Constriction Model. Intraganglionic injection in the trigeminal ganglia of AAV Isl2-KCNK4 or Syn-KCNK4 each reduced mechanical pain compared to control Isl-GFP or Syn-GFP in an infraorbital nerve constriction pain model of trigeminal neuralgia. n=10-11 mice/group with near equal representation of adult male and female animals.

### DETAILED DESCRIPTION

Human genetic evidence based on SCN9A (NaV1.7)³⁻⁷ and independent confirmation in mouse models⁸⁻¹⁰ together demonstrate that reducing firing of first order pain-sensing neurons, nociceptors, is sufficient to abrogate pain: NaV1.7 “knockout” humans have congenital insensitivity to pain but preserved other peripheral sensory modalities including touch and warmth³; in contrast, gain of function mutations in the same gene cause the rare severe pain syndromes inherited erythromelalgia^{4,6} and paroxysmal extreme pain disorder⁵. Numerous other strategies have been used to silence nociceptors with concomitant dramatic reduction in pain, supporting the strategy of reducing nociceptor firing to block pain¹¹⁻¹⁵. The overarching lesson from the human genetics is that silencing the electrical activity of nociceptors is sufficient to eliminate human pain; notably, estimates from studies using inhibitory optogenetic constructs suggest that silencing as few as 15-20% of nociceptors is sufficient to yield a marked reduction in pain¹⁵. Efforts within biotech and pharma focused on blocking or downregulating NaV1.77; however, these approaches have been met with only limited success due to the requirement for complete or near-complete block or knockdown of the target channel to sufficiently reduce excitability, the challenge in obtaining a drug that acts specifically on the human NaV1.7 channel and not other voltage gated sodium channels, and the potential need for blocking the central projections of nociceptors in the spinal cord^{10,16,17}.

Described herein is an AAV-based gene therapy strategy to overexpress a potassium channel in nociceptors to reduce pain. Potassium channels hyperpolarize neuronal membrane potential and thus decrease firing, and are down-regulated in pain models¹⁸. Human genetic evidence supports this strategy, as a gain of function mutation in KCNQ2 (Kv7.2) or KCNQ3 (Kv7.3) mitigates effects of an inherited erythromelalgia NaV1.7 mutation^{19,20} The KCNK4 channel was identified in a genetic association study as a modifier of post-surgical neuropathic pain (Blanc et al., Eur J Anaesthesiol. 2019 May; 36(5): 342-350). Similarly, small molecule activators of Kv7 channels have yielded reduced pain^{21,22}; however, Kv7 agonists suffer from prominent side effects due to the widespread expression of these channels²³.

Whereas the majority of pain complaints are actually focal, most pain treatments are systemic and are, therefore, more likely to elicit adverse effects. Thus, the development of spatially-targeted therapies provide a strategy to treat pain aggressively while minimizing side effects related to systemic treatment. In addition, described herein are small promoter segments (SProms) that provide cell-type specific payload expression but are short enough to fit together with a payload in a standard AAV viral vector. These are particularly useful in cases for which broader spatial treatment may be required, while still providing tissue-specific targeting. Thus, described herein are methods for both spatial and cell-type precision pain treatment, depending on the desired target.

### Gene Delivery Compositions

Provided herein are gene delivery compositions for use in delivering sequences encoding potassium channels for expression in neurons, preferably nociceptors.

### Potassium Channels

The present methods can include expressing a two-pore domain potassium channel (also referred to herein as two-pore potassium channels or simply potassium channels), e.g., a human channel as shown in Table A (or a homolog thereof if for use in another species). Each two-pore domain potassium channel has four transmembrane domains, two re-entrant pore (P)-forming loops, and intracellular amino- and carboxy-termini. Homo- or heterodimers create a single, central K+ selective conduction pore. See, e.g., Benarroch, Neurology September 2022, 99 (12) 516-521; Gada and Plant, Br J Pharmacol. 2019 January; 176 (2): 256-266; Czirják and Enyedi, (2002) J Biol Chem, 277 (7): 5426-3; Enyedi and Czirják, (April 2010). Physiological Reviews. 90 (2): 559-60; Lotshaw D P (2007). Cell Biochemistry and Biophysics. 47 (2): 209-56; Fink et al. (June 1998). The EMBO Journal. 17 (12): 3297-308; Baggetta et al. Two-pore domain potassium channels (K2P) in GtoPdb v.2021.3. IUPHAR/BPS Guide to Pharmacology CITE. 2021; 2021 (3) doi.org/10.2218/gtopdb/F79/2021.3.

TABLE A
Human Two-Pore Potassium Channels

| Gene | Channel | RefSeq ID | Family | Aliases |
|---|---|---|---|---|
| KCNK1 | K2p1.1 | NP_002236.1 | TWIK | TWIK-1 |
| KCNK2 | K2p2.1 | NP_001017425.2; | TREK | TREK-1 |
| | | NP_001017424.1; | | |
| | | NP_055032.1 | | |
| KCNK3 | K2p3.1 | NP_002237.1 | TASK | TASK-1 |
| KCNK4 | K2p4.1 | NP_201567.1; | TREK | TRAAK |
| | | NP_001304019.1 | | |
| KCNK5 | K2p5.1 | NP_003731.1 | TASK | TASK-2 |
| KCNK6 | K2p6 | NP_004814.1 | TWIK | TWIK-2 |
| KCNK7 | K2p7 | NP_258416.1; | TWIK | |
| | | NP_203134.1; | | |
| | | NP_203133.1; | | |
| | | NP_005705.1 | | |
| KCNK8 | | NP_004814.1; | | Does not exist |
| | | XP 047295682.1 | | |
| KCNK9 | K2p9.1 | NP_001269463.1 | TASK | TASK-3 |
| KCNK10 | K2p10.1 | NP_612191.1; | TREK | TREK-2 |
| | | NP_066984.1; | | |
| | | NP_612190.1 | | |
| KCNK11 | | NP_071753.2 | THIK | |
| KCNK12 | K2p12 | NP_071338.1 | THIK | THIK-2 |
| KCNK13 | K2p13.1 | NP_071337.2 | THIK | THIK-1 |
| KCNK14 | | NP_071753.2 | | Does not exist |
| KCNK15 | K2p15.1 | NP_071753.2 | TASK | TASK-5 |
| KCNK16 | K2p16.1 | NP_001128578.1; | TALK | TALK-1 |
| | | NP_001128579.1; | | |
| | | NP_001128577.1; | | |
| | | NP_115491.1; | | |
| | | NP_001350713.1 | | |
| KCNK17 | K2p17.1 | NP_001128583.1; | TALK | TALK-2, TASK-4 |
| | | NP_113648.2 | | |
| KCNK18 | K2p18.1 | NP_862823.1 | TRESK | TRESK |

TWIK, tandem of P-domains in a weak inward rectifying K⁺ channel; TREK, TWIK-related K⁺ channel; TASK, two-pore domain, acid-sensitive K⁺ channel; TRAAK, two-pore domain related arachidonic acid activated K⁺ channel; THIK, two-pore domain halothane inhibited K⁺ channel; TALK, two-pore domain alkaline activated K⁺ channel; and TRESK, TWIK-related spinal cord potassium channel.

In some embodiments, the potassium channel used herein is a TREK family channel, e.g., KCNK2, KCNK4, or KCNK18, preferably KCNK4 or KCNK18. In some embodiments, the two-pore potassium channel is not KCNK18.

In some embodiments of the methods and compositions described herein, variants of any of the proteins or nucleic acids described herein can also be used that are at least 80%, 85%, 90%, 95%, 97%, 98%, or 99% identical to a sequence provided herein can also be used, so long as they retain desired functionality of the parental sequence. Residues that can be changed without destroying function can be identified, e.g., by aligning similar sequences and making conservative substitutions in non-conserved regions. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm that has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### Gene Delivery Systems—Vectors and Nucleic Acids

A typical approach for in vivo introduction of nucleic acid into a living cell, particularly a call in a living animal, is by use of a viral vector containing nucleic acid, e.g., a cDNA encoding a potassium channel as described herein. Viral vectors capable of highly efficient transduction of CNS neurons may be employed in the methods described herein; vectors useful in methods of gene therapy are known in the art. Examples include adenovirus, adeno-associated virus (AAV), alphavirus, herpesvirus, retrovirus, lentivirus, or vaccinia virus vectors; any serotypes of rAAV (e.g., AAV1-AAV12) vectors, recombinant or chimeric AAV vectors, as well as other suitable viral vectors can be used. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g, can be found in Ausubel, et al., eds., Current Protocols in Molecular Biology, Greene Publishing Associates, (1989), Sections 9.10-9.14, and other standard laboratory manuals. The use of AAV vectors to deliver constructs for expression in the brain has been described, e.g., in Iwata et al., Sci Rep. 2013; 3:1472; Hester et al., Curr Gene Ther. 2009 October; 9(5): 428-33; Doll et al., Gene Therapy 1996, 3(5): 437-447; and Foley et al., J Control Release. 2014 Dec. 28; 196:71-8.

Thus, in some embodiments, the potassium channel-encoding nucleic acid is present in a vector for gene therapy, such as an AAV vector. In some instances, the AAV vector is selected from the group consisting of AAV1, AAV2, AAV3, AAV4, AAV5, AAV6, AAV7, AAV8, AAV9, AAVrh10, AAV11, and AAV12. Successful viral delivery to the DRG has been shown using AAV5 (see, e.g., Tan et al. (2015) Mol Med 21, 544-552; Bevaart et al. (2015). Hum Gene Ther Cl Dev 26, 103-112); AAV6 (see, e.g., Kaplan et al. (2014). Neuron 81, 333-348; Towne et al. (2009). Mol Pain 5, 1744-8069-5-52; Yu et al., (2019). Gene Ther 26, 308-323; Fischer et al. (2014) Gene Ther 21, 44-51; Iyer et al. (2014). Nat Biotechnol 32, 274-278. 10.1038/nbt.2834; Iyer et al. (2016). Sci Rep 6, 30570. 10.1038/srep30570); AAV8 (Foust et al. (2008). Hum Gene Ther 19, 61-70; Snyder et al. (2011). Hum Gene Ther 22, 1129-1135); AAV1, 7, 8, and 9 (Dang et al. (2017). Sci Rep-Uk 7, 927); AAV6 and AAV9 (Kudo et al. (2021). Mol Ther Methods Clin Dev 23, 11-22. 10.1016/j.omtm.2021.07.009). Delivery to DRG has also been demonstrated using AAV-PHP.S (Chakrabarti et al. (2020). Biorxiv 2020.02.08.939066; Chakrabarti et al. (2020). Arthritis Rheumatol 72, 1749-1758). Mason et al. (2010). Mol Ther 18, 715-724, compared delivery to the DRG using AAV1, 2, 3, 4, 5, 6, 8 and lentivirus, showing that AAV1, AAV5, and AAV6 performed the best, with AAV5 identified as the most effective.

A vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (e.g., Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In addition, the capsid can be altered to include one or more peptides that increase expression in the nervous system, e.g., in the CNS, see, e.g., Yao et al., Nat Biomed Eng. 2022 Oct. 10. doi: 10.1038/s41551-022-00938-7; Chatterjee et al., Gene Ther. 2022 June; 29(6): 390-397; Meng et al., Mol Ther Methods Clin Dev. 2021 Feb. 27; 21:28-41; Zhang et al., Biomaterials. 2022 February; 281:121340; Gray, Cell Gene Ther. Insights 5, 1361-1368 (2019); Nonnenmacher et al., Mol. Ther. Methods Clin. Dev. 20, 366-378 (2021) or in the peripheral nervous system (e.g., AAV-PHP.S, Chan et al., Nat Neurosci. 2017 August; 20(8): 1172-1179; AAV-MaCPNS1 and AAV-MaCPNS2, Chen et al., Neuron. 2022 Jul. 20; 110(14): 2242-2257.e6; AAV-PHP, AAV-PHP.A, AAV-PHP.B, AAV-PHP.B2, AAV-PHP.B3, AAV-PHP.eB, AAV-PHP.S, Challis et al., Nat Protoc. 2019 February; 14(2): 379-414). In some embodiments, the AAV vector is encapsulated by one of the following capsids: AAV1, AAV5, AAV6, AAV7, AAV8, AAV9, AAV-F, AAV-PHP, AAV-PHP.A, AAV-PHP.B, AAV-PHP.B2, AAV-PHP.B3, AAV-PHP.eB, AAV-PHP.S, MaCPNS1 or MaCPNS2. In some embodiments, AAV-PHP. S is used.

In some instances, a particular AAV serotype vector may be selected based upon the intended use, e.g., based upon the intended route of administration.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). High titer AAV preparations can be produced using techniques known in the art, e.g., as described in U.S. Pat. No. 5,658,776. Wild-type AAV has high infectivity and is capable of integrating into a host genome with a high degree of specificity (see, e.g., Hermonat and Muzyczka 1984 Proc. Natl. Acad. Sci., USA 81:6466-6470 and Lebkowski et al. 1988 Mol. Cell. Biol. 8:3988-3996).

Adenoviruses are a relatively well characterized group of viruses, including over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). Expressing of a transgene in the DRG has been shown using an adenoviral vector (Ma et al. (2010). Mol Pain 6, 65. 10.1186/1744-8069-6-65); a varicella-zoster virus (VZV) vector (Guedon et al. (2014). Gene Ther 21, 694-702. 10.1038/gt.2014.43); and a lentiviral vector (Mason et al. (2010). Mol Ther 18, 715-724).

As an alternative to a vector, DNA or mRNA encoding the K+ channel can also be used. DNA or mRNA can optionally be delivered in a composition with (e.g., complexed with) lipid nanoparticles (LNPs), e.g., LNPs comprising one or more cationic lipids such as 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), dimethyldioctadecylammonium bromide (DDAB), 2,3-dioleyloxy-N-[2-(sperminecarboxamido)ethyl]-N,N-dimethyl-1-propanaminium trifluoroacetate (DOSPA), 3β-[N—(N′,N′-dimethylaminoethane)-carbamoyl]cholesterol (DC-Cholesterol), (((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy) carbonyl)amino)-N,N-bis(2-hydroxyethyl)-N-methylethan-1-aminium bromide (BHEM-Cholesterol), or ethylphosphatidylcholine (ePC). The LNPs can also or alternatively comprise one or more ionizable lipids such as (2S)-2,5-bis(3-aminopropylamino)-N-[2-(dioctadecylamino) acetyl]pentanamide (DOGS; Transfectam), N¹-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol, N⁴-cholesteryl-spermine (GL67), 1,2-dilinoleyloxy-N,N-dimethyl-3-aminopropane (DLin-DMA), 2,2-dilinoleyl-4-dimethylaminoethyl-[1,3]-dioxolane (DLin-KC2-DMA), (6Z,9Z,28Z,31Z)-heptatriaconta-6,9,28,31-tetraen-19-yl 4-(dimethylamino)butanoate (DLin-MC3-DMA; MC3), lipid di((Z)-non-2-en-1-yl) 9-((4-(dimethylamino)butanoyl)oxy) heptadecanedioate (L319), heptadecan-9-yl 8-((2-hydroxyethyl) (8-(nonyloxy)-8-oxooctyl)amino) octanoate (Lipid 5), heptadecan-9-yl 8-((2-hydroxyethyl) (6-oxo-6-(undecyloxy) hexyl)amino) octanoate (Lipid H (SM-102)), and ((4-hydroxybutyl) azanediyl)bis(hexane-6,1-diyl)bis(2-hexyldecanoate) (ALC-0315). The LNPs can also or alternatively comprise one or more ionizable lipid-like materials, such as 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200), tetrakis(8-methylnonyl) 3,3′,3″,3″-(((methylazanediyl)bis(propane-3,1 diyl))bis(azanetriyl))tetrapropionate (3060ᵢ₁₀), 3,6-bis(4-(bis(2-hydroxydodecyl)amino)butyl) piperazine-2,5-dione (cKK-E12), 3,6-bis(4-(bis((9Z,12Z)-2-hydroxyoctadeca-9,12-dien-1-yl)amino)butyl) piperazine-2,5-dione (OF-02), (((3,6-dioxopiperazine-2,5-diyl)bis(butane-4,1-diyl))bis(azanetriyl))tetrakis(ethane-2,1-diyl) (9Z,9′Z,9″Z,9″Z,12Z,12′Z,12″Z,12″Z)-tetrakis(octadeca-9,12-dienoate) (OF-Deg-Lin), (((3,6-dioxopiperazine-2,5-diyl)bis(butane-4,1-diyl))bis(azanetriyl))tetrakis(butane-4,1-diyl) (9Z,9′Z,9″Z,9″Z,12Z,12′Z,12″Z,12″Z)-tetrakis(octadeca-9,12-dienoate) (OF-C4-Deg-Lin), N¹,N³,N⁵-tris(3-(didodecylamino) propyl)benzene-1,3,5-tricarboxamide (TT3), and Hexa (octan-3-yl) 9,9′,9″,9″,9″,9″-((((benzene-1,3,5-tricarbonyl)ris(azanediyl))tris(propane-3,1-diyl))tris(azanetriyl)) hexanonanoate (FTT5). In addition to cationic or ionizable lipids, LNPs can comprise one or more other lipid components, such as phospholipids (for example, phosphatidylcholine and phosphatidylethanolamine), cholesterol or polyethylene glycol (PEG)-functionalized lipids (PEG-lipids). See, e.g., Hou et al., Nature Reviews Materials volume 6, pages 1078-1094 (2021). In some embodiments, polyplex nanomicelles comprising polyethylene glycol (PEG)-polyamino acid (poly[N′—[N-(2-aminoethyl)-2-aminoethyl]aspartamide](PAsp (DET)) block copolymers are used. See, e.g., Fukushima et al., Biomaterials 270:120681 (March 2021).

Non-native regulatory sequences, gene control sequences, enhancers, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein.

Typically, the potassium channel coding region is operably linked with one or more regulatory nucleic acid components, e.g., a promoter, enhancer, or other regulatory element. A promoter included in a nucleic acid as disclosed herein can be a tissue- or cell type-specific promoter, a promoter specific to multiple tissues or cell types, an organ- or tissue-specific promoter, a promoter specific to multiple tissues or organs, a systemic or ubiquitous promoter, or a nearly systemic or ubiquitous promoter. Preferably the promoter drives expression primarily in neurons, e.g., in sensory neurons, e.g., nociceptors. Promoters having stochastic expression, inducible expression, conditional expression, or otherwise discontinuous, inconstant, or unpredictable expression are also included within the scope of the present disclosure. A promoter can include any of the above characteristics or other promoter characteristics known in the art. In some embodiments, the promoter is a promoter, e.g., a small promoter (SProm), as described herein.

Also provided herein are pharmaceutical preparations of a gene therapy construct (a nucleic acid encoding a K+ channel, e.g., in a vector or DNA and preferably linked to a promoter sequence, or an mRNA) as described herein, which can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, thus the pharmaceutical preparation can comprise one or more cells, which produce the gene delivery system.

### Promoters

In some embodiments, a polynucleotide encoding a potassium channel as described herein is operably linked to promoter suitable for expression in nociceptors. For example, a neuron subtype-specific specific promoter, such as the alpha-calcium/calmodulin kinase 2A promoter may be used to target excitatory neurons. Alternatively, a pan neuronal promoter, such as the synapsin I promoter, may be used to drive expression of the K+ channel. Other exemplary promoters include, but are not limited to, a cytomegalovirus (CMV) early enhancer/promoter; a hybrid CMV enhance/chicken β-actin (CBA) promoter; a promoter comprising the CMV early enhancer element, the first exon and first intron of the chicken β-actin gene, and the splice acceptor of the rabbit β-globin gene (commonly call the “CAG promoter”); or a 1.6-kb hybrid promoter composed of a CMV immediate-early enhancer and CBA intron 1/exon 1 (commonly called the CAGGS promoter; Niwa et al. Gene, 108:193-199 (1991)). The CAGGS promoter (Niwa et al., 1991) has been shown to provide ubiquitous and long-term expression in the brain (Klein et al., Exp. Neurol. 176:66-74 (2002)).

In some embodiments, promoters for use in the methods and compositions described herein can include promoters from a gene that is primarily or selectively expressed in DRG sensory neurons, e.g., in nociceptors. Exemplary promoters can include those from the CALCB, NGFR, POU4F1, DGKH, PP1R1C, KCND1, STAC, NEK1, ISL2, SFRP5, BEAN1, CDH19, PMP22, SCN9A, SCN10A, SCN7A, NTRK1, TRPV1, TRPV2, TRPV3, TRPV4, TRPA1, P2RX3, TRPM8, KCNJ6, ZFHX2, PRDM12, CLTCL1, DRGX, CALCA, MRGPRA3, ASIC3, and POU4F3 genes.

In some embodiments, promoters for use in the methods and compositions described herein can include short promoters (SProms) as described herein. In some embodiments, the promoters comprise the human genomic regions in Table B or as shown in SEQ ID NOs: 1-6, or comprise sequences that are at least 80%, 85%, 90%, 95%, 97%, 97.5%, 98%, or 99% identical to the genomic regions in Table B or as shown in SEQ ID NOs: 1-6. These locations are relative to GRCh38.p14 (GCF_000001405.40) also named as GRCh38/hg38.

TABLE B
Human SProm Genomic Sequence Locations

| Gene | Genomic Region | SEQ ID NO: |
|---|---|---|
| ISL2 | chr15: 76335590-76336835 | 1 |
| POU4F1 | chr13: 78603161-78604300 | 2 |
| NEK1 | chr4: 169611986-169613104 | 3 |
| PPP1R1C | chr2: 181985543-181986075 | 4 |
| NGFR | chr17: 49494446-49495491 | 5 |
| CALCB | chr11: 15072723-15073718 | 6 |

Also provided herein are nucleic acids comprising an SProm as described herein, e.g., vectors, preferably expression vectors, linked to the 5′ end of a nucleic acid encoding a heterologous polypeptide for expression of the heterologous polypeptide (e.g., a polypeptide other than the polypeptide from which the SProm is derived; for example, the ISL2 SPRom drives a sequence other than ISL2, and so on), e.g., a nucleic acid encoding a two-pore potassium channel, e.g., KCNK2, KCNK4, or KCNK18, preferably KCNK4 or KCNK18. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors include, e.g., adenovirus, adeno-associated virus (AAV), alphavirus, herpesvirus, retrovirus, lentivirus, or vaccinia virus vectors, as well as others known in the art and described herein.

### Gene Therapy for Pain

Provided herein are methods for treating pain in subjects, e.g., mammalian subjects, e.g., human or non-human veterinary subjects such as cats, dogs, rabbits, horses, cows, and goats. The pain can be, for example chronic pain, including chronic post-surgical pain and neuroma, focal nerve compression or irritation not caused by compression, including median or ulnar neuropathy, trigeminal neuralgia, occipital neuralgia, focal pains resulting from nerve injury, such as complex regional pain syndrome, post-herpetic neuralgia (shingles), focal cancer pain due to metastases, painful neuropathy (including chemotherapeutic neuropathy, diabetic neuropathy, and others), focal arthritis (which can preferably by treated by injection into or near the affected joint), myofascial pain (which can be treated by intramuscular or cutaneous injection), or chronic back pain with or without sciatica (which can be treated by intralaminar or transforaminal epidural injection or extraforaminal selective nerve root injection). The pain could also include systemic pain conditions such as fibromyalgia. In some embodiments, a therapeutically effective amount is enough to induce K+ channel expression and thus silencing in 10%, 15%, 20% or more of targeted nociceptors. The methods can also be used prophylactically as a pre-surgical treatment for high-risk procedures (e.g., amputations, mastectomy, cardiac surgery, thoracotomy, hernia repair) or high-risk subjects (e.g., elderly subjects or those with comorbidities such as addiction, dependence, hepatic or renal impairment, polypharmacy, or other conditions that might complicate the use of analgesics), to reduce the risk of post-surgical pain. “High-risk” indicates a risk above a cohort of relevant subjects. The methods include delivering a therapeutically effective amount of a nucleic acid encoding a K+ channel as described herein, e.g., linked to a promoter as described herein, e.g., in a gene therapy vector or DNA, or as simple nucleic acid, e.g., mRNA, optionally complexed with a delivery vehicle such as a lipid nanoparticle, e.g., as described herein. As used herein, a therapeutically effective amount is an amount sufficient to provide a reduction in one or more symptoms of pain in the subject, e.g., a reduction in severity or frequency of pain. A prophylactically effective amount is an amount sufficient to provide a reduction in risk of developing pain, e.g., post-surgical pain, particularly chronic post-surgical pain.

This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity, and therapeutic efficacy of the therapeutic compounds can be determined by standard pharmaceutical procedures in cell cultures or experimental animals, e.g., for determining the appropriate dose for no observed adverse event levels (NOAELs). That is the dose at which the therapeutic window is defined; see “Guidance for Industry Estimating the Maximum Safe Starting Dose in Initial Clinical Trials for Therapeutics in Adult Healthy Volunteers,” available at fda.gov/media/72309/download #:~:text=The%20NOAEL%20is%20a%20generally, (or%20asymptomatic)%20human%20volunteers. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range that includes the NOAEL dose. The dosage may vary within this range depending upon the dosage form employed and the route of administration utilized. For the present methods of the invention, a therapeutically effective dose can be estimated initially using the physiological tools described herein.

In clinical settings, the gene delivery systems for the K+ channel gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the K+ channel gene is more limited, with introduction into the subject being quite localized. For example, the gene delivery vehicle can be introduced by catheter (see U.S. Pat. No. 5,328,470) or by stereotactic injection, e.g., optionally into the cisterna magna, cerebral ventricles, or lumbar intrathecal space (e.g., Chen et al., PNAS USA 91:3054-3057 (1994)). In some embodiments, delivery methods of potassium channel-expressing virus or mRNA include intravenous, intrathecal, intracerebroventricular, intracisternal, intranerve, intra-articular, intramuscular, subcutaneous, intradermal, epidural, intracisternal, intracerebroventricular, transforaminal, selective nerve root, and stereotactic intraparenchymal administration in the dorsal root or trigeminal ganglia or spinal cord.

In some embodiments, side effects can be minimized by limiting the cell type and/or location in which the K+ channel is expressed. For example, in some embodiments, where the nucleic acid encoding a K+ channel is delivered locally to or near the site of the pain, or into or near a nerve, plexus, dorsal root ganglia, or roots innervating the site of pain (the relevant nerves can be identified by a skilled practitioner), the nucleic acid can be linked to a ubiquitous promoter, or a promoter that is cell-type specific and expressed primarily or only in the target cells (i.e., sensory neurons, e.g., nociceptors). Where the nucleic acid encoding a K+ channel is delivered systemically, the nucleic acid should preferably be linked to a promoter that is cell-type specific and expressed primarily or only in the target cells (i.e., sensory neurons, e.g., nociceptors). Examples of CNS/PNS routes of administration include intrathecal, intracisternal, intracerebroventricular, and epidural injection. Examples of systemic routes of administration include intravenous administration.

### EXAMPLES

The invention is further described in the following examples, which do not limit the scope of the invention described in the claims.

### Methods

The following materials and methods were used in the Example below.

### Cloning of AAV Plasmids:

All constructs were subcloned from pAAV-hSyn-EGFP (Addgene #50465). hSyn was replaced with SProms using the MluI and NcoI restriction digest sites. EGFP was replaced with potassium channels using the NcoI and EcoRI restriction digest sites. Potassium channels and SProms were synthesized with compatible restriction digest sites by either Twist Bioscience or Genewiz.

### AAV9 Viral Particle Delivery:

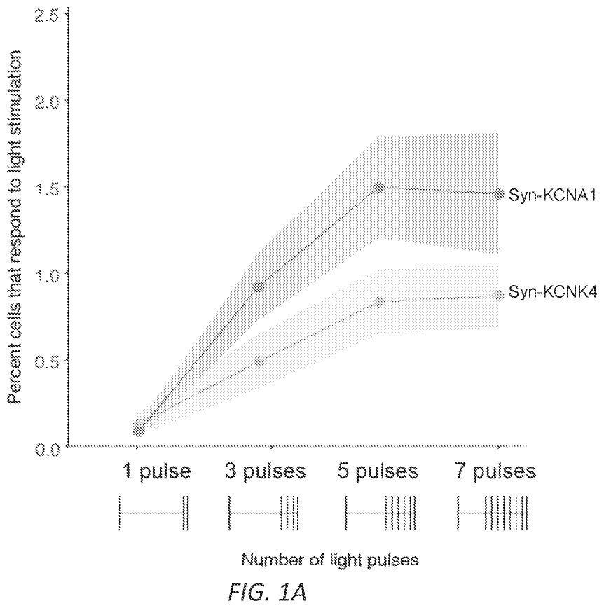

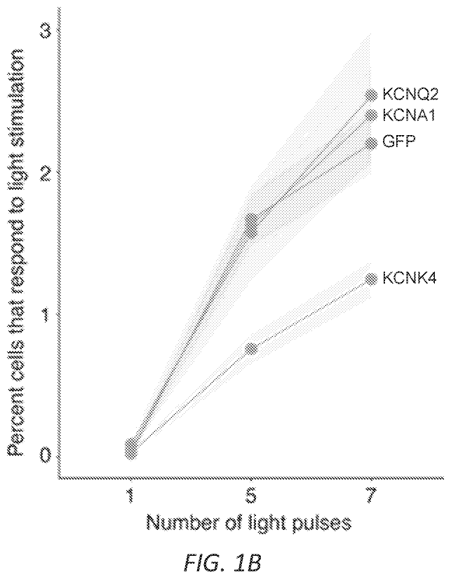

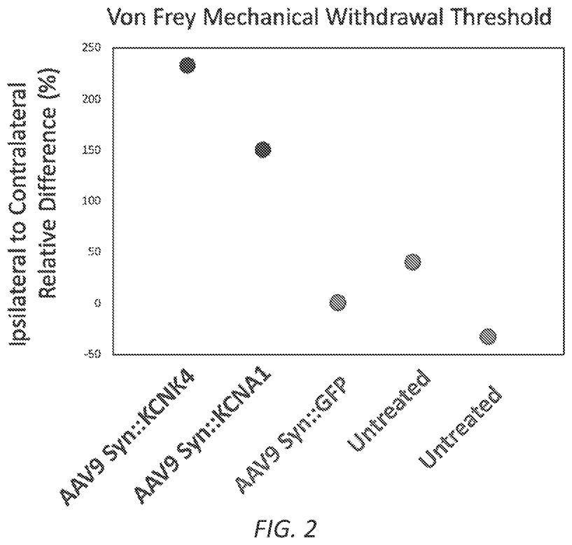

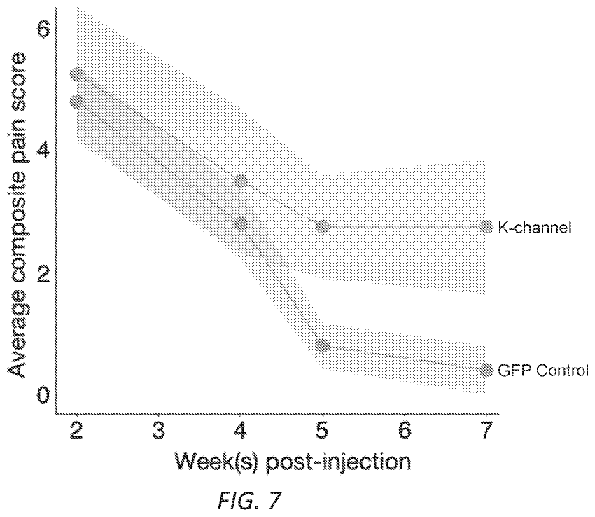

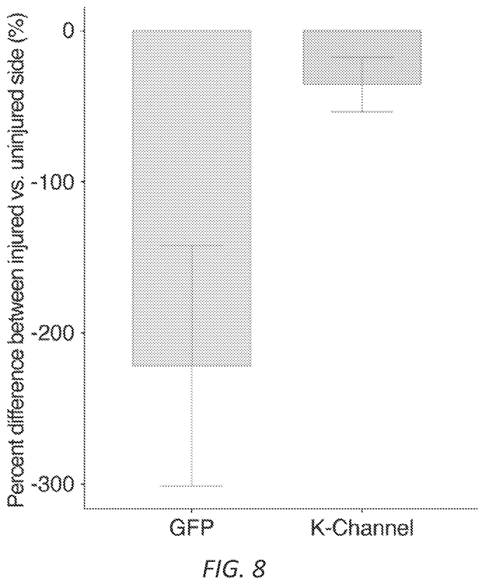

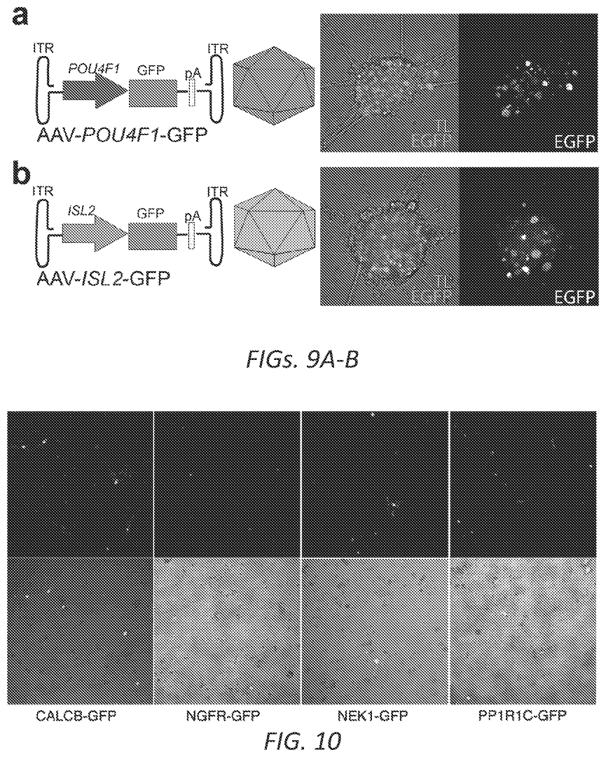

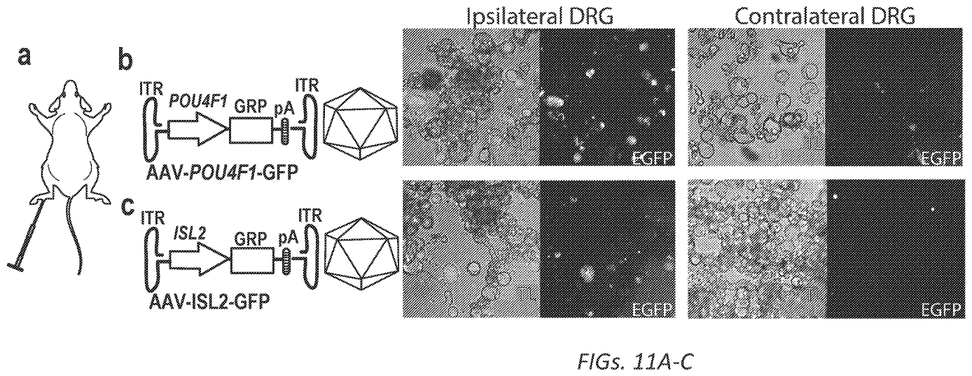

AAV9 plasmids were packaged at and titered at either the Gene Transfer Vector Core at the Grousbeck Gene Therapy Center or the Penn Vector Core. Viral particles were injected either using intraplantar injection (~1×10¹⁰ vector genomes; FIG. 11 only), intrasciatic nerve injection (~1×10¹¹ vector genomes; FIGS. 2, 4-8; FIGS. 12-14), or trigeminal ganglion injection (~1×10¹⁰ vector genomes; FIGS. 3, 15, and 18). In vitro transduction experiments of packaged vectors (FIGS. 1B and 9) were performed as described previously⁵⁶. Nucleofection of plasmids (between 1-2 μg, FIG. 10) into dissociated primary mouse DRG neurons was performed using a Lonza 4D-Nucleofector X-Unit as per company instructions.

### Patch Clamp Recording

Isolated DRG neurons were transferred to the stage of an inverted microscope (Eclipse Ti, Nikon, Tokyo, Japan). Whole-cell voltage-clamp and current-clamp recordings from small (<25 μm in apparent diameter) primary DRG neurons were conducted at 24-36 h post-dissociation, using an EPC 10 amplifier (HEKA Elektronik, Lambrecht, Germany). Thick-walled glass (Sutter Instruments, Novato, CA, USA) was pulled (P-1000, Shutter Instruments, USA) and fabricated (MF-830, Narishige, Tokyo, Japan) to form glass microelectrodes. Giga-ohm seals were achieved at room temperature (23-25° C.) with these microelectrodes. Upon establishing whole-cell configuration, cell capacitance was neutralized, and partial series resistance compensation (80%) was applied to mitigate voltage-clamp errors. The resting membrane potential (RMP) was measured in current clamp condition, and only cells with an RMP of −55 mV or lower were included in the study. Data were captured using PatchMaster software (HEKA, Germany), digitized at 20 kHz, and filtered at 5 kHz. The bath solution contained 155 mM NaCl, 3.5 mM KCl, 1.5 mM CaCl₂, 1 mM MgCl₂, 10 mM HEPES, and 10 mM glucose, pH 7.4 (NaOH). The internal solution was 140 mM K-gluconate, 13.5 mM NaCl, 1.8 mM MgCl₂, 0.09 mM EGTA, 9 mM HEPES, 14 mM creatine phosphate (Tris salt), 4 mM MgATP, and 0.3 mM Tris-GTP, pH 7.2 (KOH). Action potentials were evoked with depolarizing current steps, each 2s in duration, in 10 pA increments up to 100 pA. If no action potential was generated at 100 pA, the current was incrementally increased until the rheobase current was reached. The current rheobase was defined as the minimum current required to evoke the first action potential. Data analysis was conducted using custom scripts in MATLAB (MathWorks, USA) and OriginPro (OriginLab, USA), with results reported as mean±S.E.M.

### Sprom Identification:

Candidate promoters were selected, as described in in the results section, through a three-step process. First, genes expressed specifically in sensory neurons that have moderate to high expression were identified using RNA-sequencing and literature searches. Candidate genes from step 1 were then validated using in-situ data from the Allen Brian Atlas. Finally, we identified regulatory regions for these genes using ENCODE candidate cis-regulatory elements, histone modifications, and genetic conservation. Candidate promoter regions that passed all three steps and were less than ~1.25 kb in size were then cloned into AAV vectors for validation studies.

### Optogenetic Rheobase:

Using TrpV1::ChR2 mice, nociceptor excitability measurements using light threshold necessary to generate calcium flux in individual TrpV1-positive nociceptors was performed as described previously²⁵.

### Behavioral Models:

Spared tibial nerve injury, painful neuroma, CFA, and trigeminal neuralgia pain models were performed as described previously^{28,30,35,57,58}. Mechanical and thermal hyperalgesia were performed using Von Frey filaments and Hargreaves apparatus as we have done previously⁵⁸. Behavioral experiments were performed in approximately equal numbers of male and female adult mice.

### Data Display:

Analysis and figure generation were performed in R. Lines represent the mean and shaded areas SEMs.

### Example 1

To assess the strategy of overexpressing potassium channels, we considered members of the voltage-gated (single-pore) and two-pore potassium channel families, both of which have been implicated in pain^{18,24}. We generated AAV9 viral particles containing either the single-pore potassium channel KCNA1 (Kv1.1) or the two-pore channel KCNK4. To test channel function in vitro, we transduced Synapsin (Syn)-GFP-KCNA1 or Syn-GFP-KCNK4 (heretofore referred to as Syn-KCNA1 and Syn-KCNK4) AAV9 viral particles into primary dorsal root ganglia (DRG) neurons dissociated from TrpV1::ChR2 mice (which express the light-sensitive channelrhodopsin in TrpV1-positive nociceptors). We previously developed an optical rheobase assay of nociceptor excitability, which assesses the blue light threshold for calcium activation in individual TrpV1::ChR2 nociceptors combined via a far red calcium indicator²⁵.

After in vitro transduction, we observed that KCNK4 yielded a lower nociceptor excitability compared to KCNA1 (FIG. 1). In a pilot study to confirm this result from a behavioral perspective, intrasciatic nerve injection of AAV9 viral particles containing Syn-KCNK4 resulted in a greater increase of ipsilateral mechanical withdrawal threshold compared to Syn-KCNA1 (FIG. 2), consistent with the in vitro physiological findings.

We were particularly interested in the KCNK4 family of channels, because they are active at rest and tend to rectify either outwards or net even, allowing more favorable efflux of potassium, as opposed to members of the inward rectifier channel family⁵¹. We compared a subset of KCNK channels using in vivo trigeminal ganglion injection of AAV9 viral particles containing either a KCNK channel or GFP control into TrpV1::ChR2 mice. Four weeks after in vivo delivery, animals were sacrificed and DRG harvested and dissociated in vitro analysis using the same high-content optical platform. KCNK4, KCNK10, and KCNK18 all reduced nociceptor excitability, with largest effects observed with KCNK4 and KCNK18 (FIG. 3). Importantly, the assay reflects the entire population of TrpV1::ChR2 nociceptors, not just those transduced with the AAVs. These data show that the viral transduction is thus sufficient to exert inhibition on a large population of nociceptors using in vivo delivery. We proceeded with the KCNK4 channel for our subsequent studies.

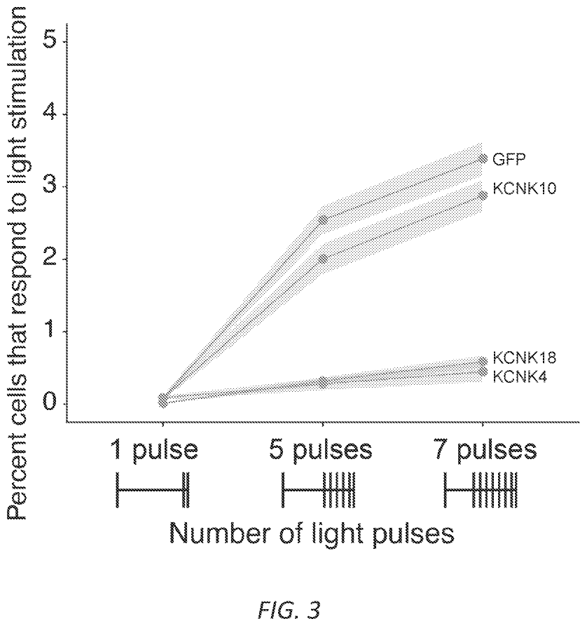

To validate our initial results, we injected AAV9 viral particles containing Syn-KCNK4 into the sciatic nerves of additional TrpV1::ChR2 mice. We found that nociceptors ipsilateral to the injection showed a marked reduction in excitability compared to control nociceptors contralateral to the injection (FIG. 4). Notably, the magnitude of this effect was large relative to those previously observed using high concentrations of drugs that reduce pain, gabapentin and retigabine, which modify alpha-2-delta calcium channel subunits or open Kv7 voltage gated potassium channels by, respectively^{23,25,26}. These results demonstrate that KCNK4 packaged in AAV9 viral particles can be delivered in vivo and reduces the excitability of nociceptors.

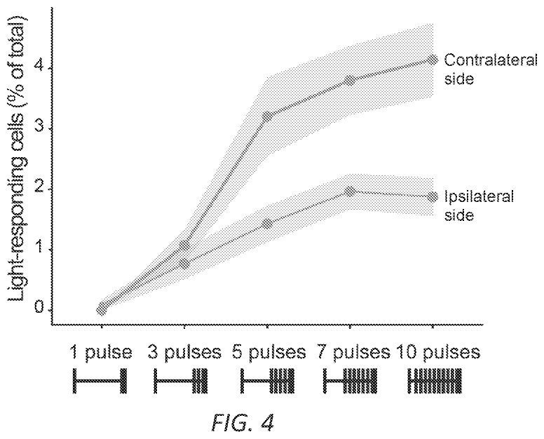

We next confirmed the physiological effect of reduction in nociceptor excitability using patch clamp. Four weeks after in vivo intrasciatic nerve injection of AAV9 Syn::GFP-KCNK4 viral particles, we sacrificed the animals and isolated and dissociated the DRG neurons. Nociceptors were identified based on small size (<20 pF capacitance), and transduced neurons were identified by GFP signal either in GFP control or GFP-KCNK4 fusion protein. Whole cell patch clamp was used to verify the electrophysiological effects of the functional KCNK4 channel protein (FIG. 5). Nociceptors expressing GFP-KCNK4 showed a profound increase in rheobase, the current threshold for action potential generation, consistent with reduction in nociceptor excitability. The cells also exhibited a reduction in input resistance, as expected for the presence of functional ion channels. Finally, the resting membrane potential was hyperpolarized, again consistent with the function of a leak potassium channel. Notably, the hyperpolarized membrane potential suggested that the transduced cells were healthy, as membrane depolarization is a common feature of sick neurons.

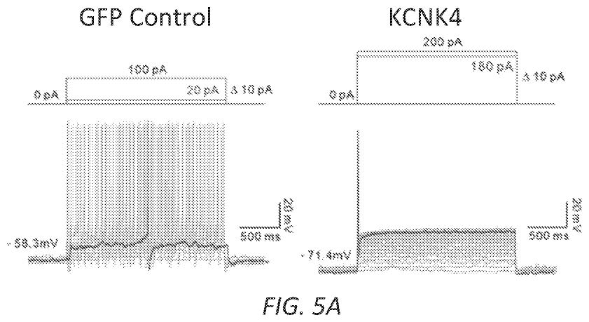

We next investigated whether AAV9 viral particles containing Syn-KCNK4 could exert effects using behavioral pain models in vivo. Nerve injury models are among the most common models of neuropathic pain²⁷. We performed the established spared tibial nerve injury model²⁸ and concomitant injection of Syn-KCNK4 or Syn-GFP control AAV9 viral particles into the proximal tibial nerve. Following the procedures, animals injected with Syn-GFP developed mechanical allodynia consistent with prior studies²⁸ (FIG. 6). In contrast, animals injected with Syn-KCNK4 viral particles had much less mechanical allodynia, consistent with a strong reduction in neuropathic pain (FIG. 6).

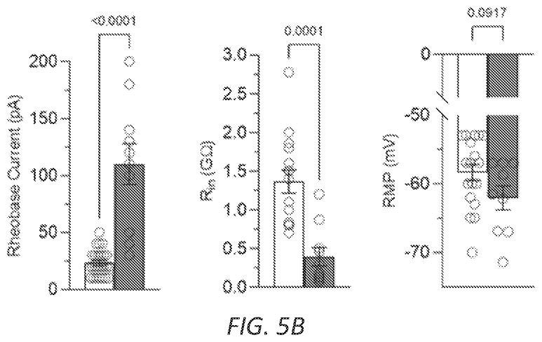

Painful neuromas are common and severe causes of pain following nerve injuries or surgeries²⁹. We used a rodent model of painful neuroma^{30,31}, and concomitant sciatic nerve injection upon sciatic neuroma induction. In this model, animals are scored based on mechanical allodynia, with baseline scores typically zero³⁰. Animals injected with Syn-GFP and Syn-KCNK4 AAV9 viral particles showed no difference in pain response for the first four weeks (FIG. 7); however at five weeks after the combined injection and pain model procedure, a time at which robust AAV expression would be expected³², there was substantial reduction in neuroma pain in animals injected with Syn-KCNK4 compared to Syn-GFP control viral particles (FIG. 7, Table 1). Because the onset of pain is rapid in the neuroma model but AAVs take several weeks to reach maximum expression^{30,33,34}, we conclude that the K channel expression may be sufficient to reduce pre-existing neuropathic pain conditions.

TABLE 1
Pain in Painful Neuroma Neuropathic Pain Model

| Week | 2 | 4 | 5 | 7 |
|---|---|---|---|---|
| GFP (n = 4) | 5.25 | 3.5 | 2.75 | 2.75 |
| K Channel (n = 5) | 4.8 | 2.8 | 0.8 | 0.4 |
| % diff | 9.3% | 25.0% | 243.8% | 588% |

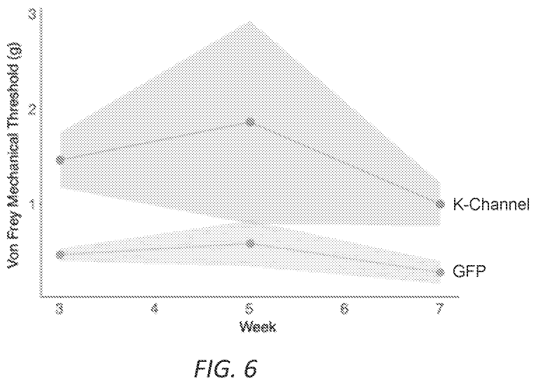

To assess the capacity for potassium channel delivery to treat inflammatory pain, we employed the commonly-used complete Freund's adjuvant (CFA) model³⁵. Because the effects of CFA injection resolve before AAV9 reaches maximum expression, we pre-injected animals with AAV9 viral particles containing either Syn-KCNK4 or control Syn-GFP in the unilateral sciatic nerves and compared behavior after subsequent intraplantar CFA injection on the same side. Whereas animals receiving control viral particles exhibited a profound thermal hyperalgesia in the ipsilateral compared to contralateral hindpaws 48 hours after CFA injection, there was almost no difference between CFA-injected and uninjected sides in animals injected with Syn-KCNK4 viral particles (FIG. 8). Thus, the minimal difference in thermal withdrawal latency in animals injected with Syn-KCNK4 indicated almost complete suppression of the inflammatory pain phenotype. Taken together, these data support the conclusion that KCNK4 expression is sufficient to reduce pain in a range of neuropathic and inflammatory pain models.

Although the synapsin promoter may be sufficient for many uses of the potassium channel gene therapy, particularly when spatial restriction is likely to be sufficient protection against potassium channel overexpression in undesired locations, we considered that some applications may require further specification. For example, across multiple delivery methods, AAVs transduce both sensory and motor neurons³⁶⁻³⁸, and the frequent side effects of potassium channel activators like the drug retigabine underscore the need for precise application³⁹. To address the need for more targeted expression, we developed short promoter sequences (SProms) that (1) were identified from genes that are strongly expressed in sensory neurons, (2) had conserved segments that were likely sufficient to preserve their function based on favorable epigenetic markers, and (3) had lengths (of such conserved segments) that were sufficiently small so as to fit together with a payload in AAV delivery vectors. Because we have focused on ion channels, we wanted to identify SProms of lengths around 1.25 kb or smaller, whereas promoter sequences often encompass >10 kb of genomic information. We first mined gene expression atlases for human (GSE3526)⁴⁰ and mouse (GSE10246)⁴¹ to identify genes with DRG specificity compared to expression in CNS as well as non-neuronal tissues. These genes were ranked based on an average DRG enrichment score for human and mouse data. To identify DRG-specific promoters for these genes, we took the top genes and developed a robust approach for promoter identification with the UCSC human genome browser using a group of annotated tracks: Open REGulatory ANNOtation (ORegAnno)⁴²; ENCODE candidate Cis Regulatory Elements (cCRE)⁴³; GeneHancer⁴⁴; and Eukaryotic Promoter Database (EPD/EPDnew)⁴⁵. We also analyzed DNAse sensitivity, sequence conservation among species, and histone acetylation tracks. Based on integrated readouts from these tracks, we identified strong candidate promoter segments for several genes with enriched expression in DRG. For initial investigation, we chose two genes, ISL2 and POU4F1 (Brn3a) (FIGS. 9A-B), and validated the DRG-specific expression in the Allen Brain Atlas as well. We then cloned the bioinformatically-identified ~1.25 kb promoter segments and positioned them upstream of a GFP coding sequence within an AAV9 vector.

We next tested the capacity of 6 of the identified SProms (Table 2) by transducing primary mouse DRG neurons in vitro with AAV9 viral particles containing either SProm POU4F1-GFP or SProm ISL2-GFP. After 14 days, we observed robust GFP expression (FIGS. 9A-B, right images). Based on the success of this strategy, we tested additional SProms for the CALB, NGFR, NEK1, and PP1R1C genes using nucleofection of primary mouse DRG neurons and observed successful SProm-driven expression (FIG. 10).

TABLE 2
Genes and short promoter segments (SProm).

| Gene | Short Promoter Segment (SPROM) | |
|---|---|---|
| ISL2 | 1,246 | bases |
| POU4F1 | 1,140 | bases |
| CALCB | 996 | bases |
| NGFR | 1,046 | bases |
| NEK1 | 1,119 | bases |
| PPP1RIC | 533 | bases |

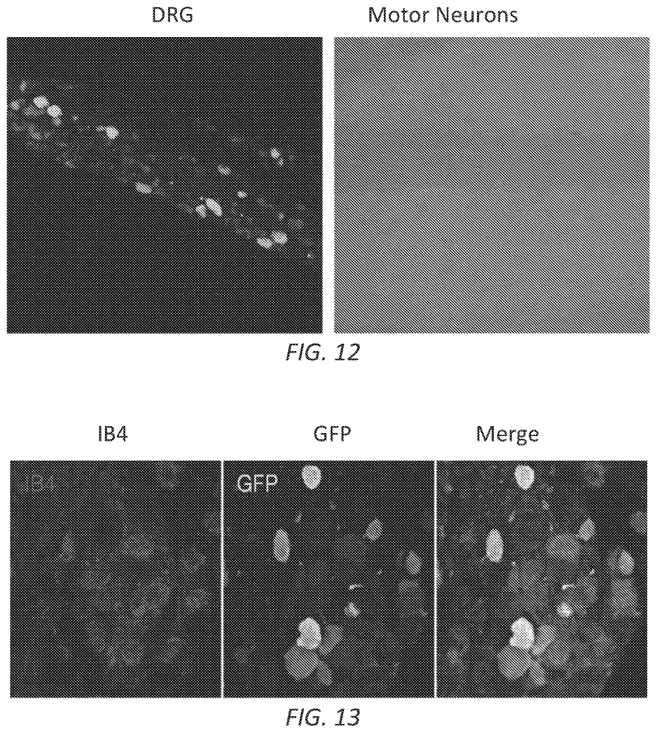

To test Sproms in vivo, we injected AAV9 viral particles containing SProm POU4F1-GFP or SProm ISL2-GFP into the hindpaws of animals via intraplantar injection, waited two weeks, euthanized the animals and dissected out the ipsilateral and contralateral DRGs from the corresponding lumbar levels (FIGS. 11A-C). Whereas the DRGs contralateral to the injected hindpaw did not show GFP expression, the ipsilateral DRGs showed robust transgene expression. We further confirmed that intrasciatic injection of AAV9 viral particles containing SProm ISL2-GFP yielded expression in ipsilateral DRG neurons, but not ipsilateral motor neurons in the ventral spinal cord, thus demonstrating the specificity obtained by using the ISL2 SProm (FIG. 12). To investigate the types of DRG neurons transduced, we used confocal microscopy and found GFP expression in DRG neurons that included IB4-positive non-peptidergic nociceptors (FIG. 13)⁴⁶.

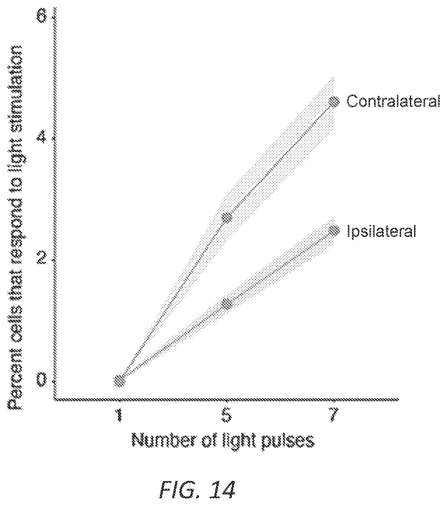

To further assess the expression driven by the ISL2 SProm as well as the efficacy of ISL2 SProm-KCNK4 in reducing nociceptor excitability, we injected AAV9 viral particles containing SProm ISL2-KCNK4 into sciatic nerves of TrpV1::ChR2 mice. Several weeks after injection, we performed the aforementioned optical rheobase assay and demonstrated that SProm ISL2-KCNK4 viral particles yielded a strong reduction in nociceptor excitability (FIG. 14), comparable to that observed with Syn-KCNK4 viral particles (FIG. 4).

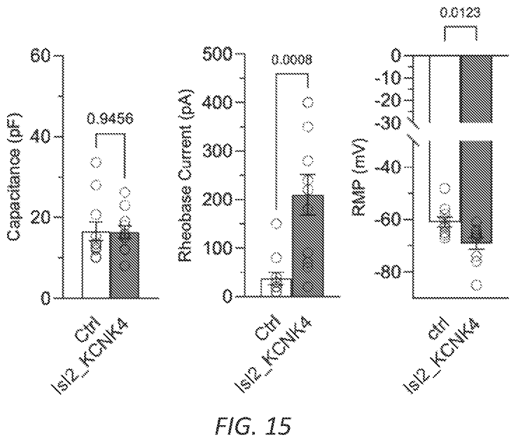

In order to confirm the functional capacity of the ISL2-GFP-KCNK4 channel, we performed patch clamp experiments following in vivo delivery as we did previously (FIGS. 5 and 15). Using trigeminal ganglion injection followed 3-4 weeks later by isolation and dissociation, the GFP signal was difficult to reliably detect in live imaging, presumably on account of the reduced strength of the promoter compared to Syn and the low abundance of channel protein. We therefore performed “blind” patch clamp experiments. Recording from small nociceptor neurons of the same capacitance (FIG. 15, left panel), we found that the ISL2-GFP-KCNK4 AAV yielded a marked increase in rheobase, demonstrating a reduction in neuronal excitability (FIG. 15, middle panel). Furthermore, hyperpolarization of the resting membrane potential was consistent with the expected function of the channel proteins (FIG. 15, right panel).

Taken together, these data demonstrate that the ISL2 SProm is capable of driving expression in a range of different nociceptor types including IB4-positive neurons, which demarcate non-peptidergic nociceptors, and TrpV1-expressing nociceptors, which are largely peptidergic⁴⁶. Furthermore, despite the expression in this large range of nociceptor types, the specificity of expression is demonstrated by the absence of motor neuron expression, as occurs using a non-specific promoter³⁶⁻³⁸.

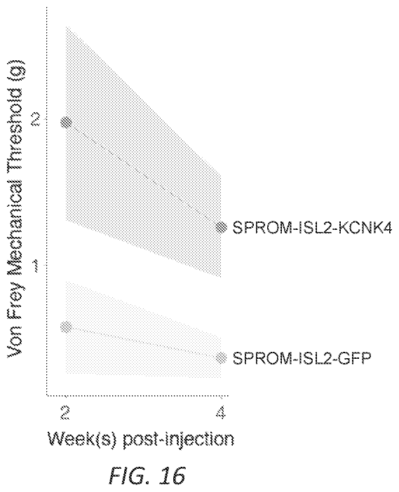

To validate the behavioral effect of SProm ISL2-KCNK4 viral particles in an animal pain behavior model, we used a neuropathic nerve injury model, as we did previously (FIG. 6). Similar to AAV9 viral particles containing Syn-KCNK4, intrasciatic injection of AAV9 viral particles containing SProm ISL2-KCNK4 yielded a profound decrease in neuropathic pain in these animals (FIG. 16). Thus, the specificity of the ISL2 promoter can be leveraged without compromising the capacity for reducing pain.

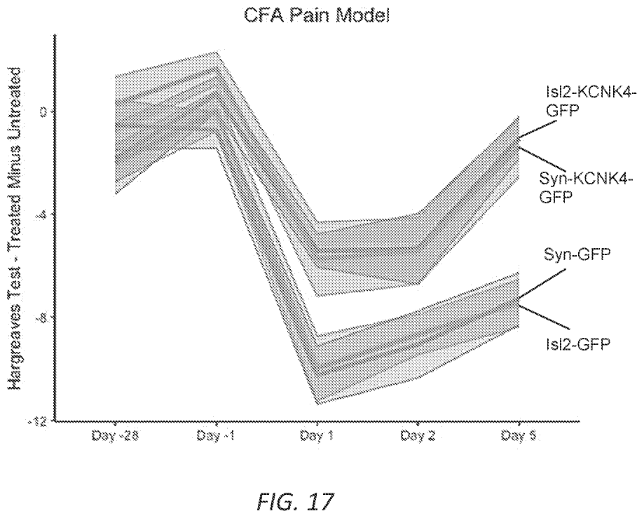

To confirm that the use of the SProm ISL2-KCNK4 channel was comparable in reducing pain to the Syn-KCNK4 channel, we performed two additional behavioral experiments in which we compared the two directly. We performed intrasciatic injection of the two different promoters driving KCNK4 channel expression, as well as a GFP control for each promoter. When we tested thermal hyperalgesia in the inflammatory CFA model, we observed a profound reduction in pain phenotype in mice injected with the KCNK4 channel AAVs (FIG. 17). Notably, the magnitude of the pain reduction was identical in the SProm ISL2 and Syn promoter viruses.

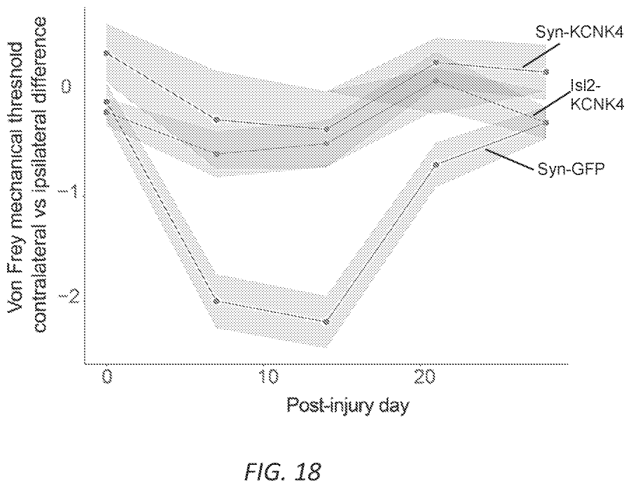

Finally, to test the expanded use of the technology, we used an infraorbital nerve constriction model of trigeminal neuralgia, a frequent and debilitating pain condition⁵⁹. We performed in vivo trigeminal ganglion injection of the same four constructs (SProm ISL2 and Syn promoters driving either KCNK4 or GFP control) followed by the infraorbital nerve constriction model (FIG. 18). Again, we observed profound reduction in pain with the KCNK4 channel, and the magnitude of the effect was similar between the SProm ISL2 and Syn promoters.

Exemplary SProm Sequences (CRCh38/hg38)

ISL2 SProm chr15: 76335590-76336835 (1246 nt)

| | |
|---|---|
| GAACCCTTTC AAACAAAACC CACAGCGATT TCTTTGAAAG AATGCCTGGA | 76335639 |
| CTGTGCTCAG AGCTCTCAGA TTTTCTCAGG ACCAACGAGC CGCCGCCTCG | 76335689 |
| CAGATACCAC TGTAAATTAC CCAGCGCCTT ACGTTCGTTT CTGATTATTT | 76335739 |
| GCATTGCAGT GGGTTTGTTA ATGAAGGACA AGAGTTTAGA AAAATCTTTT | 76335789 |
| ATTTTGGAAG TTGGACACGC AATCGACCCC AGCAGTGTTC CTAAAAGGAG | 76335839 |
| CTATGCCATT TGGATGGGGG AAAAATGAGG GGGCGGGGAA GTCCATGTTA | 76335889 |
| CATTTAAAAC AACAACAGCA ACAAATTAAA AGCAAAACAA AACAACAAAA | 76335939 |
| AAAAGGAAGT GGAGCAGGAG GAGGAGGTGG TGGTGGAGGA GAGGGAGGAG | 76335989 |
| GGGCAGCAGC AGCGGCAGCA GCAGCAAGGA TGGAGTCAGG ACCCGTCCCA | 76336039 |
| GTCCCGGGTG AGCGCTTTTG GATCTGGGTT CTGGCTCAAC CCACGTAATG | 76336089 |
| CCCCAATCTA AAGTTTTCTA CGTGTGTGTG GATAGGAAAG TCTAAATAGA | 76336139 |
| GTTTATCAGA ACCCAGGTGG TCATTCTCTA CACTCTCTCT AGCTTGGACC | 76336189 |
| CGAAGAACAA AGGCACATGA GAGGAGAAAG GTGACCAGCT GGACGATGAC | 76336239 |
| GGTCACGTCC AAACGGATCC CCCTTGTCCT CGGCTCTCTC GTCCTGAGAG | 76336289 |
| TGGGTTTAAT TTTTTTTTTT TTAATTTATA TAAGAAGGGA GCTCTTAAAG | 76336339 |
| GAGAGCCATC TCCACCCTCA GGTATCCATC ACCCAGCTCC AGCGCGCCTG | 76336389 |
| GGAAAACCGC CCCGAAAGTC CAAGAGGAAG CTCAGAGTTG TAACGGCCGC | 76336439 |
| GGAGCCAGCT CGGCGGTGAC GCAAGGTCCA GTCCAGATTG CCAGGCCCGG | 76336489 |
| GGCATGAGAG AGGATCCTTG TAGGTTTCGG AGGTGGGGGG GCTGCACTCC | 76336539 |
| ATTGTTCACT CCGGGCCAAT CAGGGTTGGC CCACTTCCTC CCAGCCAATC | 76336589 |
| TCCCTTCACC CCCAGCCTCC AACCCAACCC ACCCCGCCCA TCAGCCCCTG | 76336639 |
| GATCCCCATC ACCTCCCCCG CATCCCCGGC AGTTCTGGGG AAGCTTCGTG | 76336689 |
| ACGCCACAGG TCCCGCCCCC AGCTCCGGCC CGGGGCTAGT GCGTGTTGAC | 76336739 |
| GTCATGCTGC GTGCGGGCCG GTGCGGAATC GCTCCTTCAA CTCCGCGGGG | 76336789 |

CAGTAGGAGT TAGTTAGCAA AGAGCCGAGG CCGGGCGCGC GACCCT (SEQ ID NO: 1)
POU4F1 SProm chr13: 78603161-78604300 (1140 nt)

| | |
|---|---|
| GGGGCGGGCG CGCGGGCCGG GGCCGCGGGC GTGGGGCGCT TACCGGCGGC | 78603210 |
| GTGGGCAGGC AGGCCCGCCG GATGGCCTCG GAGCTGGAGT GCAGCGACGG | 78603260 |
| GTACTTGTGC TCAGGGAGGG TGGGATGCAT GGCAAAGTGA GGCTGCTTGC | 78603310 |
| TGTTCATGGA CATCATCGTG GCGGCTTGGC ATGTATATCC ACAAACACTC | 78603360 |
| CGAAAGTCCG CGGGAAAGTG CGTACGCCGG CTCACCCGGC CTCCCTTCGG | 78603410 |
| AGGCTGCAGC CGCGGCGGTC GCGGCGGCTG GCGGCGGCCC CGCCGCGGGC | 78603460 |
| TGCTGCTGCT GCTCCTGCTG CTGCCAGGCG CTCCCTCTGA CCGCGCAGAG | 78603510 |
| CGCCGCGCGC CGGCCTCGCG GTCCCGCTTC TCCGACAGCT CTAGCCCCGC | 78603560 |
| GCGCCGACGG GATGCACTCC TCTAACACCT GAGCCCCACT TCTCGCGGCC | 78603610 |
| GTCCCGGGGA GCTCTCGCGA GAGCTCGCGG CCCCACCGCG CTGACAGGCA | 78603660 |
| TCAGCTGTCT CCGTCTGTCT GACGCGCGCT CTCCTTCTCG GCGGCCCCGT | 78603710 |
| GCGTCTGCGC GCGCGCGCGC TCGTCCGGCC GCGACCAGCG CGTGGGAGCC | 78603760 |
| GCTCTTATAG TGACCACCAG CAAGGACAGC GCAGGTGATG CACCTGTAGC | 78603810 |
| TACCCGGGCA TGCGCACTGC CCGCCTCACC TTTCCCACCG TGGTCTGGAA | 78603860 |
| AGATCAGAAG GTCCAGCTAT TGTCTAAGGG TGGGCACCTT TTAGAATAAA | 78603910 |
| AAAGACGAGA TGCGCGCACG CCCCTCTATG TGTGTGTGTG TGTGTGTGTG | 78603960 |
| TGCGTGCGTG TGTGTGTATG TGTGTATGTT TCTTCTCCCT GATAGCAAGC | 78604010 |
| CTTGAATATA TGAGCATTAC TTAGGCATTC CTCCTCTTCG GGATGCCTAA | 78604060 |
| CAAGCTGTTG TATAATGTAT ACAGCTGGAC ATTTCTACGT TAAATTATGC | 78604110 |
| CCGCGCATTC CTCTGTATAC AGTATAAATA ACAACACAGA AATGCAGAAG | 78604160 |
| GTGCTGTCTT TTGCTGCAAG AACCTGTTTC TTTAAAGGTT TATAAATAGG | 78604210 |
| TTCCTGGAGA ATAATCGCGG TGACAGACAT TTGTTTGAAG CAGTCAGACG | 78604260 |

CTATTGATTT CCATATAATT TAATTTCCTC CGCATACTTT (SEQ ID NO: 2)
NEK1 SProm chr4: 169611986-169613104 (1119 nt)

| | |
|---|---|
| TTGCATGTCA TTCCTTCCCC CAAGTGGAAC TCACCTCAGT GACACAGGAC | 169612035 |
| GTTAGTAGGA ATAACGGTGA TGACTAATAA GCATCAGTCT AGGTGTGGCA | 169612085 |
| GTCTTCCTAC GGGGCTTAAT AAAGCAGAGG TACCATCCCG CATTTTGAAA | 169612135 |
| ATGACATCCT GAAAGAGAAA CCAAGACTGT CTAACCAGCA GCAGCTCACA | 169612185 |
| GTCCAGACTG AAAGGGGGCA CTGAGACTCT CCACACCTGA CACTCGTCTT | 169612235 |
| CAGTTGATAC CACGGGTTTG ACTCCGTCCT GTCTCGGGCG ACGCCCGTAA | 169612285 |
| GACAGGTCGA CACTACCCCG ACGAAACAAA CAAACTGGTT TCTGAGCCCC | 169612335 |
| GTAGAAACGG CGGGGTGCAG CAGGGGGAGT GCCTCCGTTA CCGCCTCTCC | 169612385 |
| AACTTCACAG AGGTCGTTGC TAGTGGCCAC GGCGGGGTGG GGACGGGCGG | 169612435 |
| CGTTCGGGAC TGGGAAGCTA CTTGACTGCC ACGTGCTCCT GCCTCGCAAC | 169612485 |
| AGCGGCTCTA GATTCCGAGT TATGGGGACA ACTGCTCGAG AGGCCAGGGT | 169612535 |
| AGGGTAAGGA CTCCGCAACC TAGGGTCCCA AAACCCTGGA GCGAATGCGG | 169612585 |
| ACTAAGGGAG AGCGGCCGGC GCGTCACCGG CGCTGGCGTG TGACGTCAGC | 169612635 |
| GCGTGGAAGG CGGCACGCAG CGGAGGCGGC TGGGAAGGAG CAGTGAGGCT | 169612685 |
| GGGCTAGAAC CGTAGGGTCG GGCGGGGTGC TGTGCGAAGG GGCGGGCGGG | 169612735 |
| GTGCTGTGCG AAGGGACGGG AGGGGCGGGC TCTGCAGGCG GGAAGACAGA | 169612785 |
| AAGGACGAGA CGGGAGAGAT GAAAAGGATG GAGAGAGTGT AAAGCTGTTA | 169612835 |
| AGAGTCATGC AGAGATGCGG GAGAGAACTC ACTAGTTGAA ACCAATTTGG | 169612885 |
| AATATAAAGC ATATAAAATT TTCCCACAGC TACTACACCG AGAAAGGGAC | 169612935 |
| TTTTTTATTT TACATGAAAG CGCACTACTT AGGGATGCCA CATTTTAAAG | 169612985 |
| AGCAATTTTG CTTTAAAAAG CAAGTCTTTT CCTATTATAT CTTGGATGGT | 169613035 |
| TGAAACAGAA GAATGTTTAA GTCTTGAGAT ATTTTTAAAA AGCTGTATGG | 169613085 |

GGTTTTCTAC ACCTTTACA (SEQ ID NO: 3)
PP1R1C SProm chr2: 181985543-181986075 (533 nt)

| | |
|---|---|
| GTGGCTGAAT GTGAAATAGC AAAGGTATCT GACTTATAAG TTGCTGAGAG | 181985592 |
| AAATGATTCC ATACCTAAAC CCTGCCCCCA ACTCTAAAAA CTTCCTGCTT | 181985642 |
| GATGTTCATC AATAATGAAT GGCCCATCCC TGCAATACTC AGAGGGAGCT | 181985692 |
| TACATTGCAC TTTTGAGCAG CCGGTTAGTA ATGTACAGTG GATAGAGCCT | 181985742 |
| TTCTTTCAAA TCCCAGGGCA GTGGGTCCTT CAAGGCTAGC ATTGCATTTT | 181985792 |
| CAATTAGCTG CTGAGTGAAT GCTTGGCATG TGTATTAGTG AAGAGGCACA | 181985842 |
| CAATTAGCGT ATTGTTCCTT TCTGTATTGT GCTGAGAGGA TCCAAGGGAT | 181985892 |
| TGGTGGGGGA ACAGGCAAGC CAGGCATCAC CGTGGATGTT GAAGAAGGGG | 181985942 |
| GTTACTCAAA CCTCGGCATC TTCACTTGCT CGATCTGGAA TTACAGCTAT | 181985992 |
| TTATTACGAA GCACTCTGTG TGGCTTAGTG GAGTGTGTCT GAGGAAACAC | 181986042 |

ATCCCGGACA CCACTTAGGG TTAGTCTTTC TGA (SEQ ID NO: 4)
NGFR SProm chr17: 49494446-49495491 (1046 nt)

| | |
|---|---|
| GTATACATGT GAGGGCTTAT GCAGACACGA CTGTATACAT GCATGTGTGA | 49494495 |
| ATGTGTACAT ATGCGCGTTT GAATGTCCGG ATCACATATG TGCCCGTGTG | 49494545 |
| CATGCCTGGA GAGGTGCATG TGTGGGACAG TCAGGTCGGC AGGAGTGCAT | 49494595 |
| GAGAACGGTG TGGGCACACG TAAGTGCACG ATCACACATA CAAGTGAGCT | 49494645 |
| TGAGAGTGTG TATTCCTGTG CACTGTGTGC ACACCTGTGA CCCCTTCAAA | 49494695 |
| CCCCTCATTT GAGAATCAAA TGATGTCGGA ATGGACTCCA CCTTGAATGT | 49494745 |
| CGATGGCCGA GGTAAGGGCG CCTGCGCCCC TCCCCTCCCC CTAGGCCTCT | 49494795 |
| GCCCAGGGAT CTCCTGCACT GGGAAGGGGA GGGGTGGGGA TGGGGTGGAG | 49494845 |
| GTGGGGTGAT GGACTGAGAT GAAGGGCAGG GAGGGCAAGC GGGCATTCTT | 49494895 |
| ACTGATAGGG GGCAGTTAGG GAGCAAGGCT CCAGGGAGAA GGTGAAGCCA | 49494945 |
| GAGGCGGAGG AAGATGGGTA AGAGAGTGAA CCCTGTGGCG GGCCGCAGCC | 49494995 |
| GGAGAGGAAC AGGAACCGCA GTGGGGACGC CCTGGTCCCC GGGCCCACAG | 49495045 |
| CATCCGGGAC GGACGCGCAG TAGCGCGGGC CGGGAACTGG GTACCAGGGC | 49495095 |
| GGGATGGGTG AGAGGCTCTA AGGGACAAGG CAGGGAGAAG CGCAGCGGGG | 49495145 |
| TGCGGGGAAC CGCACGCCCT CCCTTTGCCT CTGCTTCCCA CCCCGAGGCG | 49495195 |
| GCAGGGCGGG CGGGCGCGGT TCCGGGGGTG GGCGGGCTGG GCGGGGCGGA | 49495245 |
| GGCGGGGCCG CAGCACTGGC TTCACCCAGC CTCTCCCGCC CGCAGCCAGA | 49495295 |
| GCGAGCCGAG CCGCGGCCAG CTCCGGCGGG CAGGGGGGGC GCTGGAGCGC | 49495345 |
| AGCGCAGCGC AGCCCCATCA GTCCGCAAAG CGGACCGAGC TGGAAGTCGA | 49495395 |
| GCGCTGCCGC GGGAGGCGGG CGATGGGGGC AGGTGCCACC GGCCGCGCCA | 49495445 |

TGGACGGGCC GCGCCTGCTG CTGTTGCTGC TTCTGGGGGT GAGTGT (SEQ ID NO: 5)
CALCB SProm chr11: 15072723-15073718 (996 nt)

| | |
|---|---|
| ACCATCTCCG CCAGGCAGCT TGGCAAACAG GTGGCAGAGT TGCAGGGCAG | 15072772 |
| CTGTGTAAGC CAACTTCGGC GCAGCAGTGG AGGGTCCTGG CTTGGCGTGG | 15072822 |
| GGGATGCTGG ACCCGCGGTC GAGGATTTGG GGATATAGGG GAAGAGGGAG | 15072872 |
| GAGGTGGATG CTGAGCCTTG TATGCAGGCT ATGTCAGTTT AGCCCTCTCC | 15072922 |
| CCAACCTCTC TTCGGCTCCT GCCCGTCCCA GAGGAGTGAG GTGGAGAAGG | 15072972 |
| GCTGGCTGCC AGACTGGCAC CAAAACAGCC TTCTTTGGGT GCCCAGGTTG | 15073022 |
| CCAGGGCTCG AGGGGTCGGA GGATATCCAG GGAAGCACCC CAGGTGGTCC | 15073072 |
| AAAAAGATCA AATTTTGAGG ACCCCTCCCT CCCCTTTTCC CTCCCCCCCC | 15073122 |
| CTCCTTCCCT GCCGTGGGCT CTTTCAGCTG TGGTCCCTTT AGAACCCAGG | 15073172 |
| ACTACTACTG CTCAACCTCG CTGGGGGTTC GGGTGGCTGG ATTCGGGTCC | 15073222 |
| CTCACTGGCG TGACAGGAGG GAGTGCGAGG CAGGAATTTA GGAGCCAAGG | 15073272 |
| AGGTGAGAGC AGCTCTGGCC CCTCACTGTA GGTGACGCCA AACTCTCCTC | 15073322 |
| GACTTGCCCC GACTCTTAGT TGAAAAATCT CTGTCCTCTC CCAGGCTCTC | 15073372 |
| CAGCTTCCCA AGCAATGACC TCAATGAAAA AAATGACAGC GGGGGGACT | 15073422 |
| GCCCCCGCTC CAGAGTACCA GTGCCGGCAG TGCGAGCTAT GACGCAATCG | 15073472 |
| GAGCTCGGTC GGTCCTTTGA TTGGCTAGTC CTGGCCACTT TGGATTGGCC | 15073522 |
| GCGCGGGCTG GTGGGGACCC CCCCCCTCCA GCTATCTCTG TAATAAGAGC | 15073572 |
| GGGGTCTCCG CGGGGAAGGC GCCCACAGCA GGTGTGGTGT TCATCCCGGG | 15073622 |
| TCGACCGGCC GCTCGCGCTG CCCTGAAACT CTAGTCGCCA GGTGAGGAAC | 15073672 |

TTCCCATTCC CCATTCCGCT CACTCGAATA ACCTGCTTCT TCGACT (SEQ ID NO: 6)

### REFERENCES

1 Dahlhamer, J. et al. Prevalence of Chronic Pain and High-Impact Chronic Pain Among Adults-United States, 2016. MMWR Morb Mortal Wkly Rep 67, 1001-1006 (2018).
2 Volkow, N. D., Frieden, T. R., Hyde, P. S. & Cha, S. S. Medication-assisted therapies—tackling the opioid-overdose epidemic. N Engl J Med 370, 2063-2066 (2014).
3 Cox, J. J. et al. An SCN9A channelopathy causes congenital inability to experience pain. Nature 444, 894-898 (2006).
4 Cummins, T. R., Dib-Hajj, S. D. & Waxman, S. G. Electrophysiological properties of mutant Nav1.7 sodium channels in a painful inherited neuropathy. J Neurosci 24, 8232-8236 (2004).
5 Fertleman, C. R. et al. SCN9A mutations in paroxysmal extreme pain disorder: allelic variants underlie distinct channel defects and phenotypes. Neuron 52, 767-774 (2006).
6 Novella, S. P., Hisama, F. M., Dib-Hajj, S. D. & Waxman, S. G. A case of inherited erythromelalgia. Nat Clin Pract Neurol 3, 229-234 (2007).
7 Cao, L. et al. Pharmacological reversal of a pain phenotype in iPSC-derived sensory neurons and patients with inherited erythromelalgia. Sci Transl Med 8, 335ra356 (2016).
8 Gingras, J. et al. Global Nav1.7 knockout mice recapitulate the phenotype of human congenital indifference to pain. PLOS One 9, e105895 (2014).
9 Shields, S. D. et al. Insensitivity to Pain upon Adult-Onset Deletion of Nav1.7 or Its Blockade with Selective Inhibitors. J Neurosci 38, 10180-10201 (2018).
10 MacDonald, D. I. et al. A central mechanism of analgesia in mice and humans lacking the sodium channel Na(V)1.7. Neuron 109, 1497-1512 e1496 (2021).
11 Binshtok, A. M., Bean, B. P. & Woolf, C. J. Inhibition of nociceptors by TRPV1-mediated entry of impermeant sodium channel blockers. Nature 449, 607-610 (2007).
12 Djouhri, L., Koutsikou, S., Fang, X., McMullan, S. & Lawson, S. N. Spontaneous pain, both neuropathic and inflammatory, is related to frequency of spontaneous firing in intact C-fiber nociceptors. J Neurosci 26, 1281-1292 (2006).
13 Iyer, S. M. et al. Optogenetic and chemogenetic strategies for sustained inhibition of pain. Sci Rep 6, 30570 (2016).
14 Weir, G. A. et al. Using an engineered glutamate-gated chloride channel to silence sensory neurons and treat neuropathic pain at the source. Brain 140, 2570-2585 (2017).
15 Iyer, S. M. et al. Virally mediated optogenetic excitation and inhibition of pain in freely moving nontransgenic mice. Nat Biotechnol 32, 274-278 (2014).
16 Kingwell, K. Nav1.7 withholds its pain potential. Nat Rev Drug Discov (2019).
17 Zakrzewska, J. M. et al. Safety and efficacy of a Nav1.7 selective sodium channel blocker in patients with trigeminal neuralgia: a double-blind, placebo-controlled, randomised withdrawal phase 2a trial. Lancet Neurol 16, 291-300 (2017).
18 Du, X. & Gamper, N. Potassium channels in peripheral pain pathways: expression, function and therapeutic potential. Curr Neuropharmacol 11, 621-640 (2013).
19 Mis, M. A. et al. Resilience to Pain: A Peripheral Component Identified Using Induced Pluripotent Stem Cells and Dynamic Clamp. J Neurosci 39, 382-392 (2019).
20 Yuan, J. H. et al. KCNQ variants and pain modulation: a missense variant in Kv7.3 contributes to pain resilience. Brain Commun 3, fcab212 (2021).
21 Li, L. et al. Activation of KCNQ Channels Prevents Paclitaxel-Induced Peripheral Neuropathy and Associated Neuropathic Pain. J Pain 20, 528-539 (2019).
22 Zhang, F. et al. Antinociceptive Efficacy of Retigabine and Flupirtine for Gout Arthritis Pain. Pharmacology 105, 471-476 (2020).
23 Blackburn-Munro, G. & Jensen, B. S. The anticonvulsant retigabine attenuates nociceptive behaviours in rat models of persistent and neuropathic pain. Eur J Pharmacol 460, 109-116 (2003).
24 Mathie, A., Veale, E. L., Cunningham, K. P., Holden, R. G. & Wright, P. D. Two-Pore Domain Potassium Channels as Drug Targets: Anesthesia and Beyond. Annu Rev Pharmacol Toxicol 61, 401-420 (2021).
25 DuBreuil, D. M. et al. A high-content platform for physiological profiling and unbiased classification of individual neurons. Cell Rep Methods 1 (2021).
26 Sills, G. J. The mechanisms of action of gabapentin and pregabalin. Curr Opin Pharmacol 6, 108-113 (2006).
27 Costigan, M., Scholz, J. & Woolf, C. J. Neuropathic pain: a maladaptive response of the nervous system to damage. Annu Rev Neurosci 32, 1-32 (2009).
28 Shields, S. D., Eckert, W. A., 3rd & Basbaum, A. I. Spared nerve injury model of neuropathic pain in the mouse: a behavioral and anatomic analysis. J Pain 4, 465-470 (2003).
29 Buch, N. S., Qerama, E., Brix Finnerup, N. & Nikolajsen, L. Neuromas and postamputation pain. Pain 161, 147-155 (2020).
30 Dorsi, M. J. et al. The tibial neuroma transposition (TNT) model of neuroma pain and hyperalgesia. Pain 134, 320-334 (2008).
31 Toia, F., Giesen, T., Giovanoli, P. & Calcagni, M. A systematic review of animal models for experimental neuroma. J Plast Reconstr Aesthet Surg 68, 1447-1463 (2015).
32 Mason, M. R. et al. Comparison of AAV serotypes for gene delivery to dorsal root ganglion neurons. Mol Ther 18, 715-724 (2010).
33 Klein, R. L., Dayton, R. D., Tatom, J. B., Diaczynsky, C. G. & Salvatore, M. F. Tau expression levels from various adeno-associated virus vector serotypes produce graded neurodegenerative disease states. Eur J Neurosci 27, 1615-1625 (2008).
34 Reimsnider, S., Manfredsson, F. P., Muzyczka, N. & Mandel, R. J. Time course of transgene expression after intrastriatal pseudotyped rAAV2/1, rAAV2/2, rAAV2/5, and rAAV2/8 transduction in the rat. Mol Ther 15, 1504-1511 (2007).
35 Butler, S. H., Godefroy, F., Besson, J. M. & Weil-Fugazza, J. A limited arthritic model for chronic pain studies in the rat. Pain 48, 73-81 (1992).
36 Kaplan, A. et al. Neuronal matrix metalloproteinase-9 is a determinant of selective neurodegeneration. Neuron 81, 333-348 (2014).
37 Snyder, B. R. et al. Comparison of adeno-associated viral vector serotypes for spinal cord and motor neuron gene delivery. Hum Gene Ther 22, 1129-1135 (2011).
38 Foust, K. D., Poirier, A., Pacak, C. A., Mandel, R. J. & Flotte, T. R. Neonatal intraperitoneal or intravenous injections of recombinant adeno-associated virus type 8 transduce dorsal root ganglia and lower motor neurons. Hum Gene Ther 19, 61-70 (2008).
39 Deeks, E. D. Retigabine (ezogabine): in partial-onset seizures in adults with epilepsy. CNS Drugs 25, 887-900 (2011).
40 Roth, R. B. et al. Gene expression analyses reveal molecular relationships among 20 regions of the human CNS. Neurogenetics 7, 67-80 (2006).
41 Lattin, J. E. et al. Expression analysis of G Protein-Coupled Receptors in mouse macrophages. Immunome Res 4, 5 (2008).
42 Lesurf, R. et al. ORegAnno 3.0: a community-driven resource for curated regulatory annotation. Nucleic Acids Res 44, D126-132 (2016).
43 Consortium, E. P. et al. Expanded encyclopaedias of DNA elements in the human and mouse genomes. Nature 583, 699-710 (2020).
44 Fishilevich, S. et al. GeneHancer: genome-wide integration of enhancers and target genes in GeneCards. Database (Oxford) 2017 (2017).
45 Dreos, R., Ambrosini, G., Cavin Perier, R. & Bucher, P. EPD and EPDnew, high-quality promoter resources in the next-generation sequencing era. Nucleic Acids Res 41, D157-164 (2013).
46 Basbaum, A. I., Bautista, D. M., Scherrer, G. & Julius, D. Cellular and molecular mechanisms of pain. Cell 139, 267-284 (2009).
47 Walker, M. C. & Kullmann, D. M. Optogenetic and chemogenetic therapies for epilepsy. Neuropharmacology 168, 107751 (2020).
48 Aston-Jones, G. & Deisseroth, K. Recent advances in optogenetics and pharmacogenetics. Brain Res 1511, 1-5 (2013).
49 Wykes, R. C. et al. Optogenetic and potassium channel gene therapy in a rodent model of focal neocortical epilepsy. Sci Transl Med 4, 161ra152 (2012).
50 Ma, C., Rosenzweig, J., Zhang, P., Johns, D. C. & LaMotte, R. H. Expression of inwardly rectifying potassium channels by an inducible adenoviral vector reduced the neuronal hyperexcitability and hyperalgesia produced by chronic compression of the spinal ganglion. Mol Pain 6, 65 (2010).
51 Goldstein, S. A., Bockenhauer, D., O'Kelly, I. & Zilberberg, N. Potassium leak channels and the KCNK family of two-P-domain subunits. Nat Rev Neurosci 2, 175-184 (2001).
52 Wang, D., Tai, P. W. L. & Gao, G. Adeno-associated virus vector as a platform for gene therapy delivery. Nat Rev Drug Discov 18, 358-378 (2019).
53 Hordeaux, J. et al. Adeno-Associated Virus-Induced Dorsal Root Ganglion Pathology. Hum Gene Ther 31, 808-818 (2020).
54 Chan, K. Y. et al. Engineered AAVs for efficient noninvasive gene delivery to the central and peripheral nervous systems. Nat Neurosci 20, 1172-1179 (2017).
55 Mendell, J. R. et al. Dystrophin immunity in Duchenne's muscular dystrophy. N Engl J Med 363, 1429-1437 (2010).
56 Wainger, B. J. et al. Modeling pain in vitro using nociceptor neurons reprogrammed from fibroblasts. Nat Neurosci 18, 17-24 (2015).
57 Mogil, J. S. Animal models of pain: progress and challenges. Nat Rev Neurosci 10, 283-294 (2009).
58 Chiu, I. M. et al. Bacteria activate sensory neurons that modulate pain and inflammation. Nature 501, 52-57 (2013).
59 Ding, W. et al., An Improved Rodent Model of Trigeminal Neuropathic Pain by Unilateral Chronic Constriction Injury of Distal Infraorbital Nerve. J Pain. 18 (8): 899-907 (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating pain in a subject, the method comprising administering to the subject a nucleic acid encoding a two-pore potassium channel, optionally wherein the two-pore potassium channel is KCNK2 or KCNK4, preferably KCNK4.

2. The method of claim 1, wherein the nucleic acid encoding a two-pore potassium channel is administered by direct local injection into or near a site of pain, or a into or near a nerve, plexus, dorsal root ganglia, or roots innervating the site of pain, in the subject.

3. The method of claim 1, wherein the nucleic acid encoding a two-pore potassium channel is administered by epidural, intrathecal, or systemic administration to the subject.

4. The method of claim 1, wherein the nucleic acid encoding a two-pore potassium channel is administered as DNA or in a viral vector.

5. The method of claim 4, wherein the DNA or viral vector further comprises a promoter that drives expression of the two-pore potassium channel.

6. The method of claim 5, wherein the promoter is a ubiquitous promoter or a tissue specific promoter that preferentially drives expression in neurons, preferably in sensory neurons, preferably in nociceptors, preferably a synapsin promoter or a promoter as shown in SEQ ID NOs: 1-6, optionally wherein the two-pore potassium channel is KCNK2, KCNK4, or KCNK18, preferably KCNK4 or KCNK18.

7. The method of claim 6, wherein the promoter is ISL2 SProm.

8. The method of claim 4, wherein the viral vector is an AAV, preferably AAV5, AAV6, or AAV9.

9. The method of claim 1, wherein the nucleic acid encoding a two-pore potassium channel is administered as mRNA, preferably in a composition comprising a deliver vehicle, optionally a lipid nanoparticle.

10. A nucleic acid comprising a sequence at least 80% identical to a sequence as shown in SEQ ID NOs: 1-6, linked to a nucleic acid encoding a heterologous polypeptide for expression of the heterologous polypeptide.

11. An expression vector comprising the nucleic acid of claim 10.

12. (canceled)

13. The expression vector of claim 11, wherein the viral vector is an AAV, preferably AAV5, AAV6, or AAV9.

14. The expression vector of claim 11, wherein the heterologous polypeptide is a potassium channel, preferably a two-pore potassium channel.

15. The nucleic acid of claim 14, wherein the two-pore potassium channel is KCNK2, KCNK4, or KCNK18, preferably KCNK4 or KCNK18.

16. The nucleic acid or expression vector of claim 14, for use in a method of treating pain in a subject.

17. (canceled)

18. A cell comprising the nucleic acid or expression vector of claim 10, and optionally expressing the heterologous polypeptide.

19. An AAV vector comprising a nucleic acid encoding a KCNK4 or KCNK18 potassium channel, linked to a promoter for expression of the KCNK4 or KCNK18 potassium channel in sensory neurons.

20. The AAV vector of claim 19, which is an AAV9 vector.

21. The AAV vector of claim 19, comprising a synapsin promoter or a promoter as shown in SEQ ID NOs: 1-6, or comprising a sequence at least 80% identical to a sequence as shown in SEQ ID NOs: 1-6.

22. The AAV vector of claim 21, wherein the promoter is ISL2 SProm or comprises a sequence at least 80% identical to an ISL2 SProm sequence as shown in SEQ ID NOs: 1-6.

23. The AAV vector of claim 19, comprising a nucleic acid encoding a KCNK18 potassium channel, linked to a promoter for expression of the KCNK18 potassium channel in sensory neurons, wherein the promoter comprises a sequence at least 80% identical to a sequence as shown in SEQ ID NOs: 1-6.

24.-25. (canceled)
